(19) United States
(12) Patent Application Publication
(10) Pub. No.: US 2026/0315720 A1
(43) Pub. Date: Oct. 15, 2026

(54) CONSTRUCTION OF TUBES FOR USE WITH POSITIONING AND STABILISING STRUCTURES FOR PATIENT INTERFACES AND RESPIRATORY PRESSURE THERAPY DEVICES

(71) Applicant: ResMed Asia Pte. Ltd., Singapore (SG)

(72) Inventors: Lina GIRDAUSKAITE, Sydney (AU); Skye Kimberley SHARMA, Sydney (AU); Thontira SUPAOPASPHUN, Singapore (SG)

(21) Appl. No.: 19/484,818

(22) PCT Filed: May 17, 2024
(86) PCT No.: PCT/AU2024/050495

§ 371 Date: Nov. 14, 2025

(30) Foreign Application Priority Data
May 18, 2023 (AU) 2023901533

(51) Int. Cl. A61M 16/08; A61M 16/06; A61M 25/00

(52) U.S. Cl. CPC A61M 16/0875; A61M 16/0683; A61M 2025/0004; A61M 2207/00

(57) ABSTRACT

A patient interface including a positioning and stabilising structure with gas delivery tubes providing for flow of pressurised air from a connection port to a seal-forming structure around the patient's airways. The gas delivery tubes are constructed or otherwise configured with an outer textile layer and an impermeable inner layer that forms the interior. In an example, the impermeable layer may comprise an inner textile layer which is knitted to the outer textile layer or a silicone or polymer layer applied to the outer textile layer. The technology may alternatively be used to construct or otherwise configure at least a portion of the air circuit between a patient interface and the respiratory pressure therapy device.

### 1 CROSS-REFERENCE TO RELATED APPLICATIONS

This application claims priority to Australian Patent Application No. 2023901533, filed 18 May 2023, which is hereby incorporated by reference herein in its entirety.

### 2 BACKGROUND OF THE TECHNOLOGY

### 2.1 Field of the Technology

The present technology relates to one or more of the screening, diagnosis, monitoring, treatment, prevention and amelioration of respiratory-related disorders. The present technology also relates to medical devices or apparatus, and their use.

### 2.2 Description of the Related Art

### 2.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Certain disorders may be characterised by particular events, e.g. apneas, hypopneas, and hyperpneas.

Examples of respiratory disorders include Obstructive Sleep Apnea (OSA), Cheyne-Stokes Respiration (CSR), respiratory insufficiency, Obesity Hypoventilation Syndrome (OHS), Chronic Obstructive Pulmonary Disease (COPD), Neuromuscular Disease (NMD) and Chest wall disorders.

A range of therapies have been used to treat or ameliorate such conditions. Furthermore, otherwise healthy individuals may take advantage of such therapies to prevent respiratory disorders from arising. However, these have a number of shortcomings.

### 2.2.2 Therapies

Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders.

### 2.2.2.1 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass). Examples of such respiratory therapies include Continuous Positive Airway Pressure (CPAP) therapy, for treatment of OSA and the like, as well as non-invasive ventilation (NIV), which provides ventilatory support for patients requiring treatment for CSR and other respiratory disorders, and invasive ventilation (IV), used for patients unable to breath for themselves. Devices used for these therapies may have one of more of the following issues: uncomfortable, difficult to use, expensive, aesthetically unappealing, and/or ineffective.

### 2.2.3 Respiratory Therapy Systems

These respiratory therapies may be provided by a respiratory therapy system or device. Such systems and devices may also be used to screen, diagnose, or monitor a condition without treating it.

A respiratory therapy system may comprise a Respiratory Pressure Therapy Device (RPT device), an air circuit, a humidifier, a patient interface, an oxygen source, and data management.

### 2.2.3.1 Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH₂O relative to ambient pressure. For other forms of therapy, such as the delivery of oxygen, the patient interface may not include a seal sufficient to facilitate delivery to the airways of a supply of gas at a positive pressure of about 10 cmH₂O. For flow therapies such as nasal HFT, the patient interface is configured to insufflate the nares but specifically to avoid a complete seal. Examples of such a patient interface include a nasal cannula, nasal masks, and full-face masks, among others.

Certain other mask systems may be functionally unsuitable for the present field. For example, purely ornamental masks may be unable to maintain a suitable pressure. Mask systems used for underwater swimming or diving may be configured to guard against ingress of water from an external higher pressure, but not to maintain air internally at a higher pressure than ambient.

2.2.3.1.1 Seal-forming structure

Patient interfaces may include a seal-forming structure. Since it is in direct contact with the patient's face, the shape and configuration of the seal-forming structure can have a direct impact the effectiveness and comfort of the patient interface.

A patient interface may be partly characterised according to the design intent of where the seal-forming structure is to engage with the face in use. In one form of patient interface, a seal-forming structure may comprise a first sub-portion to form a seal around the left naris and a second sub-portion to form a seal around the right naris. In one form of patient interface, a seal-forming structure may comprise a single element that surrounds both nares in use. Such single element may be designed to for example overlay an upper lip region and a nasal bridge region of a face. In one form of patient interface a seal-forming structure may comprise an element that surrounds a mouth region in use, e.g. by forming a seal on a lower lip region of a face. In one form of patient interface, a seal-forming structure may comprise a single element that surrounds both nares and a mouth region in use. These different types of patient interfaces may be known by a variety of names by their manufacturer including nasal masks, full-face masks, nasal pillows, nasal puffs and oro-nasal masks.

A seal-forming structure that may be effective in one region of a patient's face may be inappropriate in another region, e.g. because of the different shape, structure, variability and sensitivity regions of the patient's face. For example, a seal on swimming goggles that overlays a patient's forehead may not be appropriate to use on a patient's nose.

Certain seal-forming structures may be designed for mass manufacture such that one design is able to fit and be comfortable and effective for a wide range of different face shapes and sizes. To the extent to which there is a mismatch between the shape of the patient's face, and the seal-forming structure of the mass-manufactured patient interface, one or both must adapt in order for a seal to form.

One type of seal-forming structure extends around the periphery of the patient interface, and is intended to seal against the patient's face when force is applied to the patient interface with the seal-forming structure in confronting engagement with the patient's face. The seal-forming structure may include an air or fluid filled cushion, or a moulded or formed surface of a resilient seal element made of an elastomer such as a rubber. With this type of seal-forming structure, if the fit is not adequate, there will be gaps between the seal-forming structure and the face, and additional force will be required to force the patient interface against the face in order to achieve a seal.

Another type of seal-forming structure incorporates a flap seal of thin material positioned about the periphery of the mask so as to provide a self-sealing action against the face of the patient when positive pressure is applied within the mask. Like the previous style of seal forming portion, if the match between the face and the mask is not good, additional force may be required to achieve a seal, or the mask may leak. Furthermore, if the shape of the seal-forming structure does not match that of the patient, it may crease or buckle in use, giving rise to leaks.

Another type of seal-forming structure may comprise a friction-fit element, e.g. for insertion into a naris, however some patients find these uncomfortable.

A range of patient interface seal-forming structure technologies are disclosed in the following patent applications: WO 1998/004310; WO 2006/074513; WO 2010/135785.

### 2.2.3.1.2 Positioning and Stabilising Structure

A seal-forming structure of a patient interface used for positive air pressure therapy is subject to the corresponding force of the air pressure to disrupt a seal. Thus a variety of techniques have been used to position the seal-forming structure, and to maintain it in sealing relation with the appropriate portion of the face. Several factors may be considered when comparing different positioning and stabilising techniques. These include: how effective the technique is at maintaining the seal-forming structure in the desired position and in sealed engagement with the face during use of the patient interface; how comfortable the interface is for the patient; whether the patient feels intrusiveness and/or claustrophobia when wearing the patient interface; and aesthetic appeal.

One technique is the use of adhesives. See for example US Patent Application Publication No. US 2010/0000534. However, the use of adhesives may be uncomfortable for some. Another technique used is one or more straps and/or stabilising harnesses. Many such harnesses suffer from being one or more of ill-fitting, bulky, uncomfortable and awkward to use.

Some existing headgear is formed with a predefined shape. A flat component may be shaped to a predefined, 3D shape. In other words, the flat component may be shaped so that it extends in directions along different planes. But in doing so, creases or wrinkles are created. Some existing headgear may be rigidised or supported to hold the predefined shape. For instance, the headgear may include support structures or rigidisers provided to a headgear material or materials e.g. textiles or fabrics. The support structures or rigidisers have a predefined shape or are formed into a shape so that headgear material adopts the shape of the support structure or rigidiser. The headgear material does not have a predefined shape. Therefore, the support structure or rigidiser may help the headgear material hold its shape. However, the headgear material can crease or wrinkle as it is bent by the rigidiser or support structure

### 2.2.3.1.3 Pressurised Air Conduit

In one type of treatment system, a flow of pressurised air is provided to a patient interface through a conduit in an air circuit that fluidly connects to the patient interface at a location that is in front of the patient's face when the patient interface is positioned on the patient's face during use. The conduit may extend from the patient interface forwards away from the patient's face.

2.2.3.1.4 Pressurised Air Conduit used for Positioning/Stabilising the Seal-Forming Structure

Another type of treatment system comprises a patient interface in which a tube that delivers pressurised air to the patient's airways also functions as part of the headgear to position and stabilise the seal-forming portion of the patient interface at the appropriate part of the patient's face. This type of patient interface may be referred to as having “conduit headgear” or “headgear tubing”, and the tubes referred to as “conduit tubes” or “headgear tubes”. Such patient interfaces allow the conduit in the air circuit providing the flow of pressurised air from a respiratory pressure therapy (RPT) device to connect to the patient interface in a position other than in front of the patient's face. One example of such a treatment system is disclosed in US Patent Publication No. US 2007/0246043, the contents of which are incorporated herein by reference, in which the conduit connects to a tube in the patient interface through a port positioned in use on top of the patient's head.

Conduit tubes may be formed from biocompatible plastics material, such as medical grade silicone or the like. It is desirable for patient interfaces incorporating headgear tubing to be comfortable for a patient to wear over a prolonged duration when the patient is asleep, form an air-tight and stable seal with the patient's face, while also able to fit a range of patient head shapes and sizes. For comfort, a textile layer or sleeve may be added to the conduit tube. However, these can slip relative to the tube, and create an uncomfortable wearing experience for the patient. Conduit tubes desirably should be cost-effective for manufacture but the addition of textiles can complicate the production process.

### 2.2.3.2 Respiratory Pressure Therapy (RPT) Device

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus RPT devices may also act as flow therapy devices. Examples of RPT devices include a CPAP device and a ventilator.

The designer of a device may be presented with an infinite number of choices to make. Design criteria often conflict, meaning that certain design choices are far from routine or inevitable. Furthermore, the comfort and efficacy of certain aspects may be highly sensitive to small, subtle changes in one or more parameters.

2.2.3.3 Air circuit

An air circuit is a conduit or a tube constructed and arranged to allow, in use, a flow of air to travel between two components of a respiratory therapy system such as the RPT device and the patient interface. In some cases, there may be separate limbs of the air circuit for inhalation and exhalation. In other cases, a single limb air circuit is used for both inhalation and exhalation.

Conduits used for air circuits may be constructed from biocompatible plastics material, such as medical grade silicone or the like. In use, these may create drag across bedding as the patient moves, particularly when the air circuit is relatively long. The air circuit may also add weight and tension to the patient interface, by virtue of its weight, particularly when the patient is seated and the air circuit is unsupported and suspended between the patient interface and the RPT device.

### 2.2.3.4 Humidifier

Delivery of a flow of air without humidification may cause drying of airways. The use of a humidifier with an RPT device and the patient interface produces humidified gas that minimizes drying of the nasal mucosa and increases patient airway comfort. In addition, in cooler climates, warm air applied generally to the face area in and about the patient interface is more comfortable than cold air.

### 2.2.3.5 Data Management

There may be clinical reasons to obtain data to determine whether the patient prescribed with respiratory therapy has been “compliant”, e.g. that the patient has used their RPT device according to one or more “compliance rules”. One example of a compliance rule for CPAP therapy is that a patient, in order to be deemed compliant, is required to use the RPT device for at least four hours a night for at least 21 of 30 consecutive days. In order to determine a patient's compliance, a provider of the RPT device, such as a health care provider, may manually obtain data describing the patient's therapy using the RPT device, calculate the usage over a predetermined time period, and compare with the compliance rule. Once the health care provider has determined that the patient has used their RPT device according to the compliance rule, the health care provider may notify a third party that the patient is compliant.

There may be other aspects of a patient's therapy that would benefit from communication of therapy data to a third party or external system.

Existing processes to communicate and manage such data can be one or more of costly, time-consuming, and error-prone.

2.2.3.6 Vent technologies

Some forms of treatment systems may include a vent to allow the washout of exhaled carbon dioxide. The vent may allow a flow of gas from an interior space of a patient interface, e.g., the plenum chamber, to an exterior of the patient interface, e.g., to ambient.

Polysomnography (PSG) is a conventional system for diagnosis and monitoring of cardio-pulmonary disorders, and typically involves expert clinical staff to apply the system. PSG typically involves the placement of 15 to 20 contact sensors on a patient in order to record various bodily signals such as electroencephalography (EEG), electrocardiogramalectrooculograpy (EOG), electromyography (EMG), etc. PSG for sleep disordered breathing has involved two nights of observation of a patient in a clinic, one night of pure diagnosis and a second night of titration of treatment parameters by a clinician. PSG is therefore expensive and inconvenient. In particular, it is unsuitable for home screening/diagnosis/monitoring of sleep disordered breathing.

Screening and diagnosis generally describe the identification of a condition from its signs and symptoms. Screening typically gives a true/false result indicating whether or not a patient's SDB is severe enough to warrant further investigation, while diagnosis may result in clinically actionable information. Screening and diagnosis tend to be one-off processes, whereas monitoring the progress of a condition can continue indefinitely. Some screening/diagnosis systems are suitable only for screening/diagnosis, whereas some may also be used for monitoring.

Clinical experts may be able to screen, diagnose, or monitor patients adequately based on visual observation of PSG signals. However, there are circumstances where a clinical expert may not be available, or a clinical expert may not be affordable. Different clinical experts may disagree on a patient's condition. In addition, a given clinical expert may apply a different standard at different times.

### 3 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

A first aspect of the present technology relates to apparatus used in the screening, diagnosis, monitoring, amelioration, treatment or prevention of a respiratory disorder.

Another aspect of the present technology relates to methods used in the screening, diagnosis, monitoring, amelioration, treatment or prevention of a respiratory disorder.

An aspect of certain forms of the present technology is to provide methods and/or apparatus that improve the compliance of patients with respiratory therapy.

One form of the present technology comprises a positioning and stabilising structure configured to provide a force to hold the seal-forming structure in a therapeutically effective position on the patient's head. The positioning and stabilising structure includes at least one strap.

One form of the present technology comprises a patient interface comprising a plenum chamber, a seal-forming structure, and a positioning and stabilising structure.

One form of the present technology comprises a patient interface comprising a plenum chamber pressurisable to a therapeutic pressure of at least 4 cmH2O above ambient air pressure. The plenum chamber includes at least one plenum chamber inlet port sized and structured to receive a flow of air at the therapeutic pressure for breathing by a patient. The patient interface also comprises a seal-forming structure that is constructed and arranged to form a seal with a region of the patient's face surrounding an entrance to the patient's airways. The seal-forming structure has a hole therein such that the flow of air at said therapeutic pressure is delivered to at least an entrance to the patient's nares. The seal-forming structure is constructed and arranged to maintain said therapeutic pressure in the plenum chamber throughout the patient's respiratory cycle in use. The patient interface also comprises a positioning and stabilising structure to provide a force to hold the seal-forming structure in a therapeutically effective position on the patient's head. The positioning and stabilising structure comprises at least one conduit tube configured with an internal passage for pressurised air to flow. The conduit tube is configured with a first end, arranged to engage or otherwise connect with a connection port for a respiratory pressure therapy (RPT) device, and a second end, arranged to engage or otherwise connect to the plenum chamber.

One form of the present technology comprises a conduit tube for use in an air circuit that connects a respiratory pressure therapy (RPT) device which is configured to generate a flow of air at a positive pressure above ambient, and a patient interface configured to receive the flow of air, and deliver the flow of air to one or more airways of a patient for treatment of a respiratory disorder, wherein the conduit tube has a hollow interior for the flow of air to pass therethrough from the RPT device to the patient interface, wherein the hollow interior is defined by a layer of impermeable material, the conduit tube comprising:
an outer textile layer; and
an inner layer that forms the layer of impermeable material, wherein the inner layer permeates at least partially into the outer textile layer.

In examples, the outer textile layer comprises a plurality of loops of yarn in a knitted structure.

In examples, the inner layer has a contact surface and an inner surface, the inner surface defining the hollow interior of the conduit tube.

In examples, the contact surface of the inner layer permeates at least partially through and/or around the loops of yarn of the knitted structure.

According to another aspect of the present technology, there is provided a method of manufacturing a conduit tube for use in an air circuit that connects a respiratory pressure therapy (RPT) device which is configured to generate a flow of air at a positive pressure above ambient, and a patient interface configured to receive the flow of air, and deliver the flow of air to one or more airways of a patient for treatment of a respiratory disorder, wherein the conduit tube has a hollow interior for the flow of air to pass therethrough from the RPT device to the patient interface, wherein the hollow interior is defined by a layer of impermeable material, the method comprising the following steps:
a) forming an outer textile layer;
b) applying the layer of impermeable material to a side of the outer textile layer; and
c) implementing a phase change in the layer of impermeable material such that it permeates at least partially into the outer textile layer.

In examples, there may be a further step of implementing a second phase change in the layer of impermeable material to bond it to the outer textile layer.

One form of the present technology comprises a conduit tube for use in an air circuit that connects a respiratory pressure therapy (RPT) device which is configured to generate a flow of air at a positive pressure above ambient, and a patient interface configured to receive the flow of air, and deliver the flow of air to one or more airways of a patient for treatment of a respiratory disorder, wherein the conduit tube has a hollow interior for the flow of air to pass therethrough from the RPT device to the patient interface, wherein the hollow interior is defined by a layer of impermeable material, the conduit tube comprising:
an outer textile layer; and
an inner layer that forms the layer of impermeable material, wherein the inner layer comprises a phase change material, and wherein the inner layer is applied to the outer textile layer in a first phase condition and is bonded to the outer textile layer in a second phase condition.

One form of the present technology comprises a conduit tube for a positioning and stabilising structure configured to hold a seal-forming structure of a patient interface in a sealing position on a patient's face for delivery of a flow of air to one or more airways of the patient for treatment of a respiratory disorder, wherein the conduit tube has a hollow interior for a flow of air to pass therethrough, wherein the hollow interior is defined by a layer of impermeable material, the conduit tube comprising:
an outer textile layer; and
an inner layer that forms the layer of impermeable material, wherein the inner layer comprises a phase change material, and wherein the inner layer is applied to the outer textile layer in a first phase condition and is bonded to the outer textile layer in a second phase condition.

According to another aspect of the present technology, there is provided a method of manufacturing a conduit tube, as described herein, the method comprising the following steps:
a) providing a textile layer which has a first surface that, in use, forms the outer surface of the conduit tube;
b) applying a phase change material to a second surface of the textile layer, wherein the phase change material is in a first phase condition; and
c) implementing a change in the phase change material from the first phase condition to a second phase condition, wherein, when the phase change material is in the second phase condition, the phase change material forms the layer of impermeable material defining the hollow interior of the conduit tube.

Another form of the present technology comprises a conduit tube for a positioning and stabilising structure configured to hold a seal-forming structure of a patient interface in a sealing position on a patient's face for delivery of a flow of air to one or more airways of the patient for treatment of a respiratory disorder, wherein the conduit tube has a hollow interior for a flow of air defined by a layer of impermeable material, the conduit tube comprising:
a first textile layer; and
a second textile layer, wherein the second textile layer is knitted to the first textile layer and forms the layer of impermeable material of the hollow interior of the conduit tube.

According to another aspect of the present technology there is provided a patient interface, wherein the patient interface comprises:
a plenum chamber pressurisable to a therapeutic pressure of at least 6 cmH₂O above ambient air pressure, wherein the plenum chamber comprises an inlet configured to receive a flow of air at the therapeutic pressure for breathing by a patient;
a seal-forming structure configured to form a seal with a region of the patient's face surrounding an entrance to the patient's airways, wherein the seal-forming structure is configured to maintain said therapeutic pressure in the plenum chamber throughout the patient's respiratory cycle in use; and
a positioning and stabilising structure comprising at least one conduit tube having a hollow interior defined by a layer of impermeable material, and wherein the conduit tube is configured and arranged to receive the flow of air at a first end and deliver said flow of air to the inlet of the plenum chamber at a second end, wherein the conduit tube comprises:
a first textile layer; and
a second textile layer, wherein the second textile layer is knitted to the first textile layer and forms the layer of impermeable material of the hollow interior of the conduit tube.

In examples, the first textile layer forms the outer surface or the patient contacting side of the conduit tube.

In examples, the first textile layer may be a breathable textile.

In an example, the conduit tube may comprise a plated knit structure. For instance, the conduit tube may comprise the first textile layer and the second textile layer as a plated knit structure.

In an example, the plated knit structure may comprise a technical face and a technical back, wherein the technical face may be formed by a first yarn comprising the first textile layer, and the technical back may be formed by a second yarn comprising the second textile layer, and wherein the first textile layer and the second textile layer may be knitted together.

In an example, the plated knit structure comprises one or more yarns as a series of loops.

In examples, the yarn of the first textile layer may be a synthetic yarn or natural yarn, or a blend thereof. The yarn may be selected for its physical properties, such as colour, ability to retain colour, toughness, longevity, cross-sectional profile or any combination thereof.

In a non-limiting example, the yarn of the first textile layer may comprise polyamide material. In one such example, the first layer may comprise of a yarn of nylon.

In a non-limiting example, the yarn of the first textile layer may comprise polyurethane material. In one such example, the first layer may comprise a yarn of polyurethane co-polymer material such as elastane (e.g. spandex, Lycra®, or ROICA™) or the like.

In examples, the yarn of the second textile layer is a phase changing polymer yarn.

Throughout the present specification, reference to the term “phase change” should be understood to mean a material that can change from a first phase condition/state to a second phase condition/state. For example, the material may change from a solid phase to a semi-solid state upon a change of temperature, for example, upon application of heat. Conversely, the material may change from a liquid or semi-solid state to a solid state upon a change of temperature, for example, upon application of a coolant.

In a non-limiting example, the yarn of the second textile layer is one of polypropylene, polyethylene, thermoplastic polyurethane, and a thermoplastic elastomer, or a blend thereof. However, other polymer yarns with the desired phase change properties may also be used for the yarn of the second textile layer.

In examples, the second textile layer is activated, i.e. undergoes a phase change during manufacture of the conduit tube.

In examples, the phase change is stimulated or initiated through application of heat, chemicals, or a mechanical process after the plated knit structure has been formed.

According to another aspect of the present technology, there is provided a method of manufacturing a conduit tube, as described herein, the method comprising the following steps:
a) knitting a first yarn to a second yarn to form a plated knit structure, having a technical face and a technical back;
b) implementing a first phase change of the first yarn; and
c) implementing a second phase change of the first yarn.

In an example, the first yarn dominates the yarn forming the technical back, i.e. the inner surface of the conduit tube, and the second yarn forms the technical face, i.e. the outer surface of the conduit tube.

In an example, step b) comprises applying a chemical agent to at least a portion of the first yarn dominating the technical back to stimulate the first phase change from a solid to at least a semi-solid.

In this example, step c) comprises applying a neutralising agent to at least a portion of the first yarn dominating the technical back to stimulate the second phase change from at least a semi-solid to a solid. In an alternative example, step c) comprises effecting a change in the temperature of the portion of the first yarn dominating the technical back to stimulate the second phase change from at least a semi-solid to a solid.

In an example, step b) comprises positioning the plated knit structure on a shaping component with a predefined shape, e.g. a mould or a mandrel with a predefined shape.

In this example, step b) also comprises the plated knit structure being heated by the shaping component to a temperature at which the first yarn may undergo at least a partial phase change for a time period. For instance, the conduit tube can be heated by a mandrel to a temperature at which the first yarn will become molten.

In an example, step c) comprises cooling the shaping component to a temperature at which the molten first yarn may undergo at least a partial phase change, allowing the first yarn to at least partially integrate and bond to the second yarn. For instance, step c) comprises the knitted plate structure being cooled by the shaping component to a temperature at which the second textile layer will set/harden to a solid, and in doing so bond with the first textile layer.

In an example, step b) comprises applying radiation heating to the plated knit structure to stimulate the first phase change from a solid to at least a semi-solid.

In an example, step c) comprises applying a cooling agent to the plated knit structure. For instance, the cooling agent may be chilled air or another gas.

In an example, step b) comprises applying ultraviolet light to the plated knit structure to stimulate the first phase change from a solid to at least a semi-solid.

In an example, step c) comprises applying a cooling agent to the plated knit structure. For instance, the cooling agent may be chilled air or another gas.

Another form of the present technology comprises a conduit tube for a positioning and stabilising structure configured to hold a seal-forming structure of a patient interface in a sealing position on a patient's face, wherein the conduit tube has a hollow interior for a flow of air defined by a layer of impermeable material, the conduit tube comprising:
a textile layer; and
a polymer layer, wherein the polymer layer of impermeable material defines the hollow interior of the conduit tube.

In examples, the textile layer forms the outer surface or the patient contacting side of the conduit tube.

In an example, the textile layer may comprise a plated knit structure of a yarn. In some examples, the plate knit structure may comprise of a first yarn and a second yarn.

In an example, the textile layer may comprise a plated knit structure of one or more yarns as a series of loops.

In an example, the plated knit structure may comprise a technical face and a technical back.

In examples, the yarn of the plated knit structure may be a synthetic yarn or natural yarn, or a blend thereof. The yarn may be selected for its physical properties, such as colour, ability to retain colour, toughness, longevity, cross-sectional profile or any combination thereof.

In a non-limiting example, the yarn of the plated knit structure may be comprised of a polyamide material. In one such example, the plated knit structure may be comprised of a yarn of nylon.

In a non-limiting example, the yarn of the plated knit structure may be comprised of a polyurethane material. In one such non-limiting example, the plated knit structure may be comprised of a yarn of polyurethane co-polymer material such as elastane (e.g. spandex, Lycra®, or ROICA™) or the like.

In an example, the layer of polymer may be comprised of a phase changing material.

In a non-limiting example, the layer of polymer may be comprised of a silicone rubber of an appropriate grade. In another non-limiting example, the layer of polymer may be comprised of one or more of a polyurethane (PU), a thermoplastic polyurethane (TPU), a thermoplastic elastomer (TPE), a biodegradable thermoplastic polyurethane (bTPU). In yet another non-limiting example, the layer of polymer may be a silane-polyisobutylene (Si-PIB) with one or more of the preceding polymers.

In an example, an additional layer of polymer may be provided, such that a first layer of polymer acts as an adhesive for a second layer of polymer.

According to another aspect of the present technology, there is provided a method of manufacturing a conduit tube as described herein, the method comprising the following steps:
a) knitting a plated knit structure having a technical face and a technical back;
b) applying a layer of polymer to the technical back, wherein the layer of polymer is in a first phase condition;
c) placing the layer of polymer into a second phase condition, thereby forming the impermeable layer of the conduit tube.

In an example, step b) may comprise spraying or coating the technical back of the plated knit structure with the layer of polymer.

In this example, step c) may comprise applying heat or cooling to the plated knit structure.

In an example, step b) may comprise dipping the technical back of the plated knit structure into the layer of polymer.

In this example, step c) may comprise applying heat or cooling to the plated knit structure.

In an example, step b) may comprise positioning the plated knit structure on a shaping component with a predefined shape, e.g. a mould or a mandrel with a predefined shape, wherein the technical back contacts the shaping component and wherein the polymer has been applied to the shaping component.

In this example, step c) may comprise heating or cooling the shaping component to place the polymer into a second phase condition.

In an example, step b) may, prior to application of the polymer, comprise positioning the plated knit structure on a shaping component with a predefined shape, e.g. a mould or a mandrel with a predefined shape, wherein the technical face of the plate knit structure contacts the shaping component. In this example, there may be an additional step comprising inverting the plated knit structure during or after step c).

In an example, step b) may comprising application of two or more layers of polymer. In this example, one of the layers of polymer may be selected to act as an adhesive for a second layer of polymer that forms the impermeable layer.

One form of the present technology comprises a conduit tube for a positioning and stabilising structure configured to hold a seal-forming structure of a patient interface in a sealing position on a patient's face, wherein the conduit tube has a hollow interior for a flow of air to pass therethrough, wherein the hollow interior is defined by a layer of impermeable material, the conduit tube comprising: an outer textile layer; and an inner layer that forms the layer of impermeable material, wherein the inner layer permeates at least partially into the outer textile layer.

In examples, the outer textile layer comprises a plurality of loops of yarn in a knitted structure.

In examples, the inner layer has a contact surface and an inner surface, the inner surface defining the hollow interior.

In examples, the contact surface of the inner layer permeates at least partially through and/or around the loops of yarn of the knitted structure.

According to another aspect of the present technology, there is provided a method of manufacturing a conduit for a positioning and stabilising structure configured to hold a seal-forming structure of a patient interface in a sealing position on a patient's face, wherein the conduit tube has a hollow interior for a flow of air to pass therethrough, wherein the hollow interior is defined by a layer of impermeable material, the method comprising the following steps:
a) forming an outer textile layer;
b) applying the layer of impermeable material to a side of the outer textile layer; and
c) implementing a phase change in the layer of impermeable material such that it permeates at least partially into the outer textile layer.

In examples, there may be a further step of implementing a second phase change in the layer of impermeable material to bond it to the outer textile layer.

Another aspect of the present technology is a conduit for use in an air circuit that connects a respiratory pressure therapy (RPT) device which is configured to generate a flow of air at a positive pressure above ambient, and a patient interface configured to receive the flow of air, and deliver the flow of air to one or more airways of a patient for treatment of a respiratory disorder, wherein the conduit has a wall which defines a hollow interior through which the flow of air passes in use, and wherein the wall of the conduit is substantially air impermeable and has an outer surface which comprises a textile.

In examples the textile may comprise a first yarn, and a second yarn, wherein the first yarn is knitted to the second yarn.

In examples, the first yarn may comprise a breathable or otherwise air permeable material.

In examples, the first yarn may be a synthetic yarn, a natural yarn, or a blend of synthetic and natural yarns.

In examples, the second yarn may comprise a phase changing polymer.

In examples, the second yarn may comprise a polyamide material, a nylon, a polyurethane, or a co-polymer such as elastane.

In examples, the first yarn and the second yarn may be knitted to form a textile with a plated knit structure.

In examples, the plated knit structure may comprise a technical face and a technical back, wherein the technical face is provided by the first yarn, and the technical back is provided by the second yarn.

In examples, the conduit may comprise an inner layer of substantially impermeable material and an outer layer of textile, the inner layer being attached to the outer layer.

In examples, the inner layer may permeate at least partially into the outer layer.

In examples, the inner layer may comprise a phase change material.

In examples, the inner layer may comprise a yarn.

In examples, the yarn of the inner layer may be one of polypropylene, polyethylene, thermoplastic polyurethane, and a thermoplastic elastomer, or a blend thereof.

In examples, the outer textile layer may comprise a plurality of loops of yarn in a knitted structure.

In examples, the outer textile layer may comprise a synthetic yarn, a natural yarn, or a blend of synthetic and natural yarns.

Another aspect of one form of the present technology is a patient interface that is moulded or otherwise constructed with a perimeter shape which is complementary to that of an intended wearer.

An aspect of one form of the present technology is a method of manufacturing apparatus.

Another aspect of one form of the present technology is a method of assembling a modular system comprising selecting a positioning and stabilising structure, and connecting the positioning and stabilising structure to either a first cushion or a second cushion.

An aspect of certain forms of the present technology is a medical device that is easy to use, e.g. by a person who does not have medical training, by a person who has limited dexterity, vision or by a person with limited experience in using this type of medical device.

An aspect of one form of the present technology is a portable RPT device that may be carried by a person, e.g., around the home of the person.

An aspect of one form of the present technology is a patient interface that may be washed in a home of a patient, e.g., in soapy water, without requiring specialised cleaning equipment. An aspect of one form of the present technology is a humidifier tank that may be washed in a home of a patient, e.g., in soapy water, without requiring specialised cleaning equipment.

The methods, systems, devices and apparatus described may be implemented so as to improve the functionality of a processor, such as a processor of a specific purpose computer, respiratory monitor and/or a respiratory therapy apparatus. Moreover, the described methods, systems, devices and apparatus can provide improvements in the technological field of automated management, monitoring and/or treatment of respiratory conditions, including, for example, sleep disordered breathing.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 4 BRIEF DESCRIPTION OF THE DRAWINGS

The present technology is illustrated by way of example, and not by way of limitation, in the figures of the accompanying drawings, in which like reference numerals refer to similar elements including:

### 4.1 Respiratory Therapy Systems

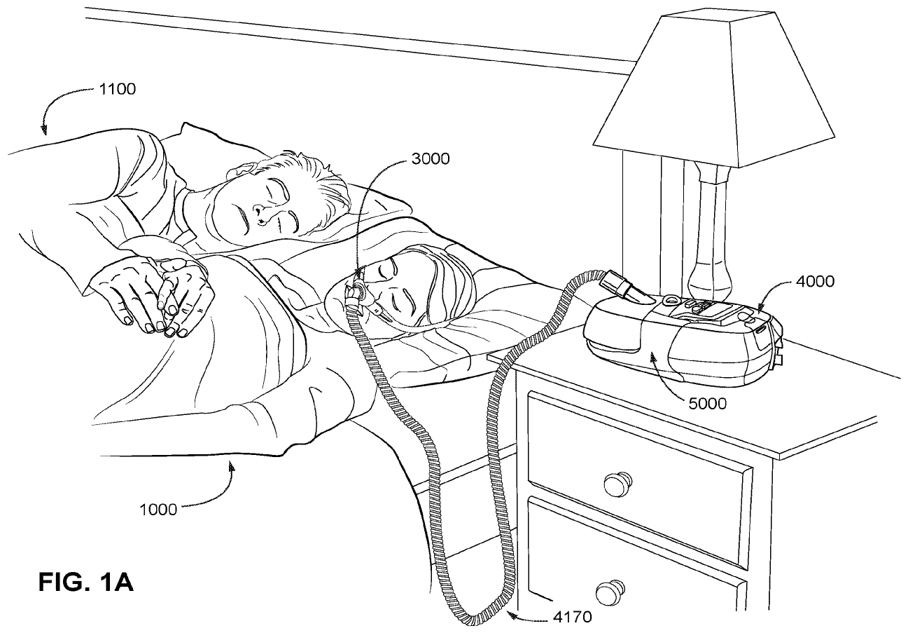

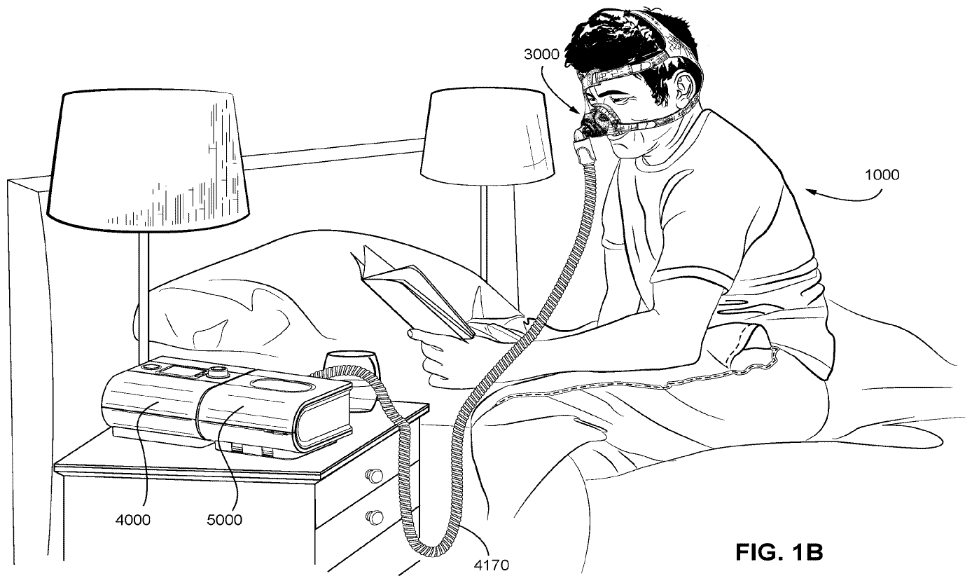

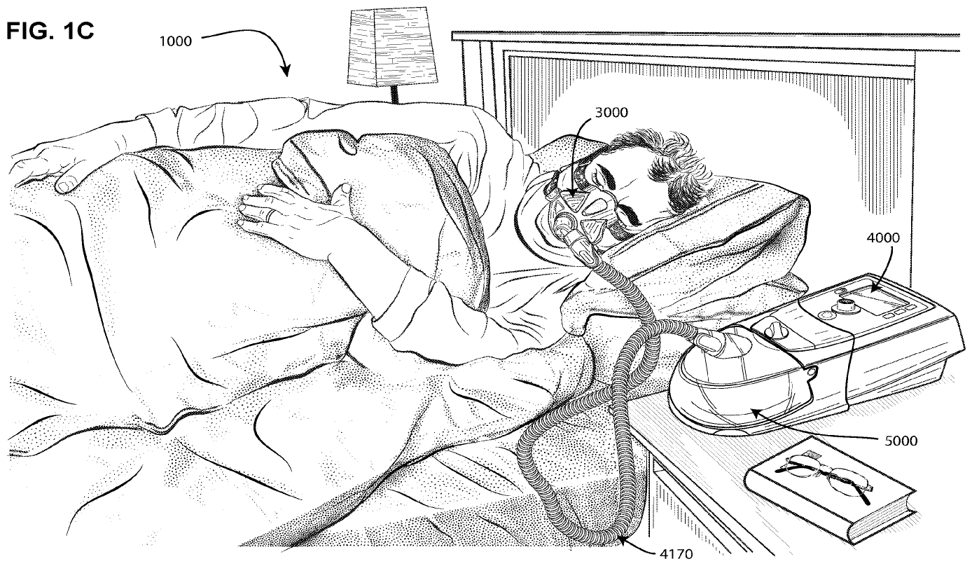

FIG. 1A shows a system including a patient 1000 wearing a patient interface 3000, in the form of nasal pillows, receiving a supply of air at positive pressure from an RPT device 4000. Air from the RPT device 4000 is humidified in a humidifier 5000, and passes along an air circuit 4170 to the patient 1000. A bed partner 1100 is also shown. The patient is sleeping in a supine sleeping position.

FIG. 1B shows a system including a patient 1000 wearing a patient interface 3000, in the form of a nasal mask, receiving a supply of air at positive pressure from an RPT device 4000. Air from the RPT device is humidified in a humidifier 5000, and passes along an air circuit 4170 to the patient 1000.

FIG. 1C shows a system including a patient 1000 wearing a patient interface 3000, in the form of a full-face mask, receiving a supply of air at positive pressure from an RPT device 4000. Air from the RPT device is humidified in a humidifier 5000, and passes along an air circuit 4170 to the patient 1000. The patient is sleeping in a side sleeping position.

### 4.2 Respiratory System and Facial Anatomy

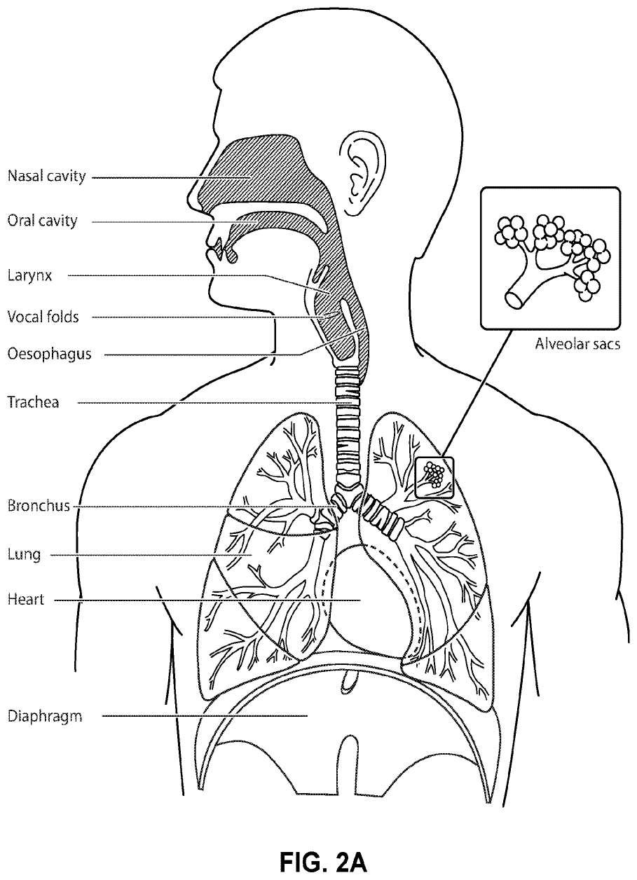

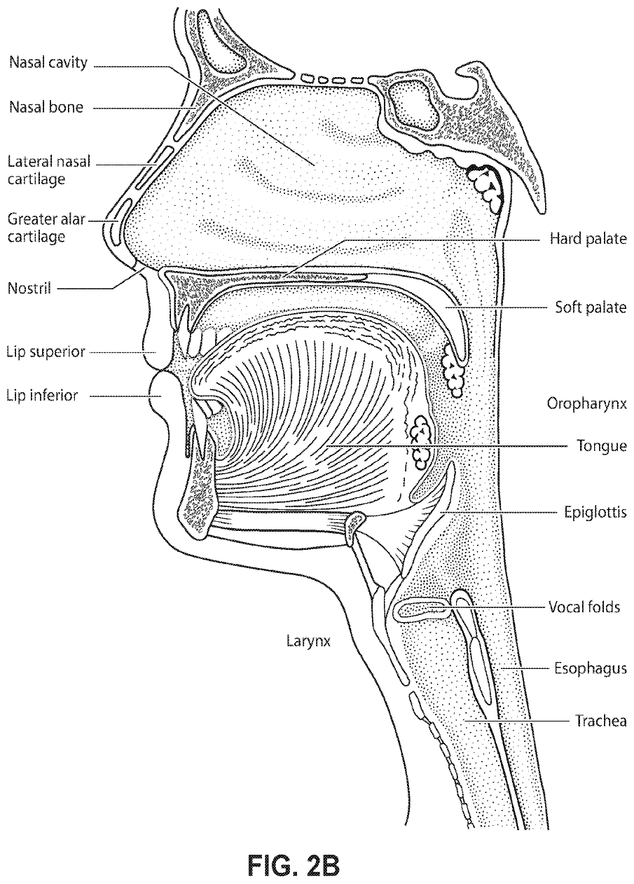

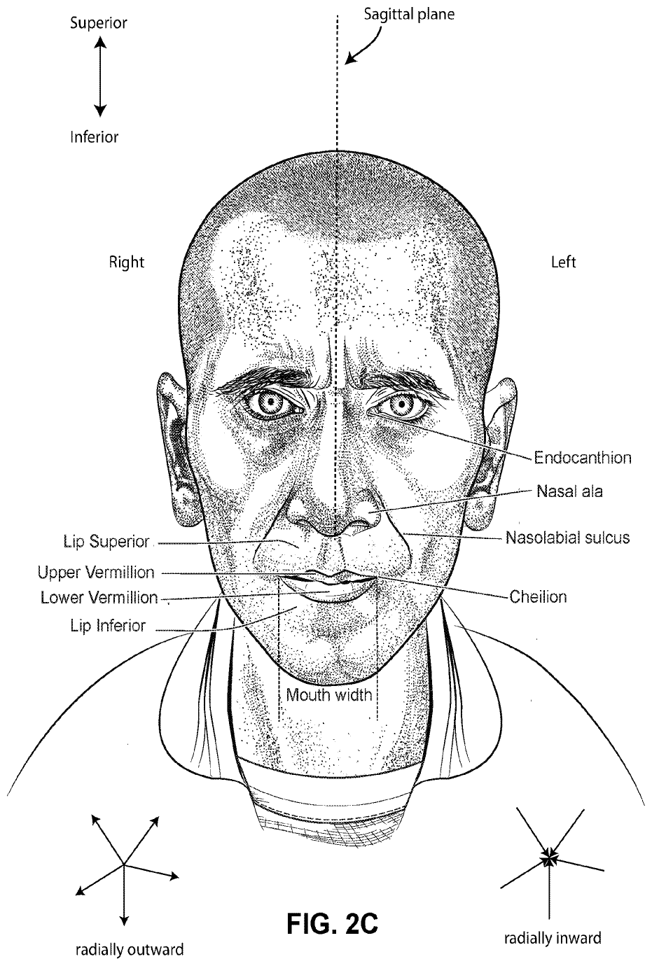

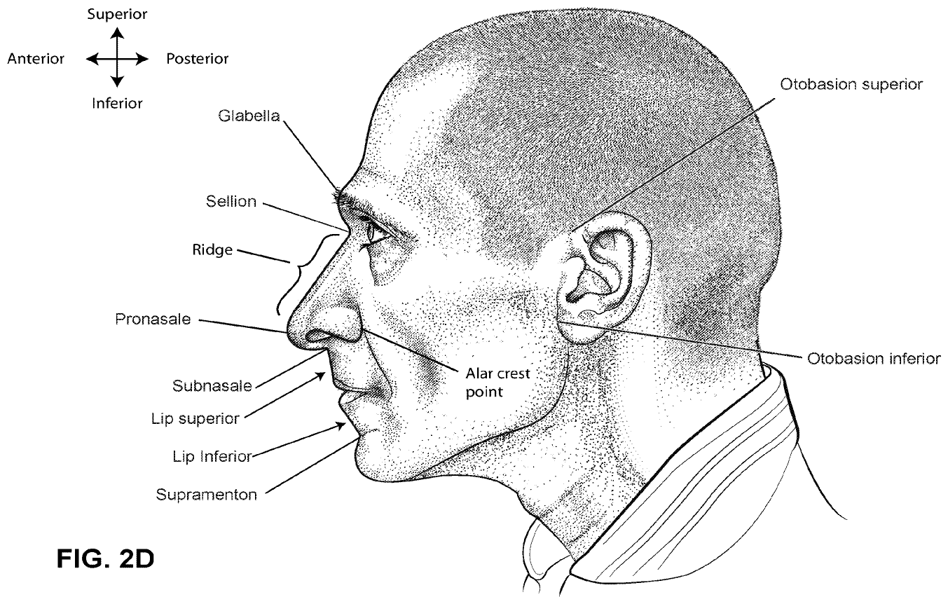

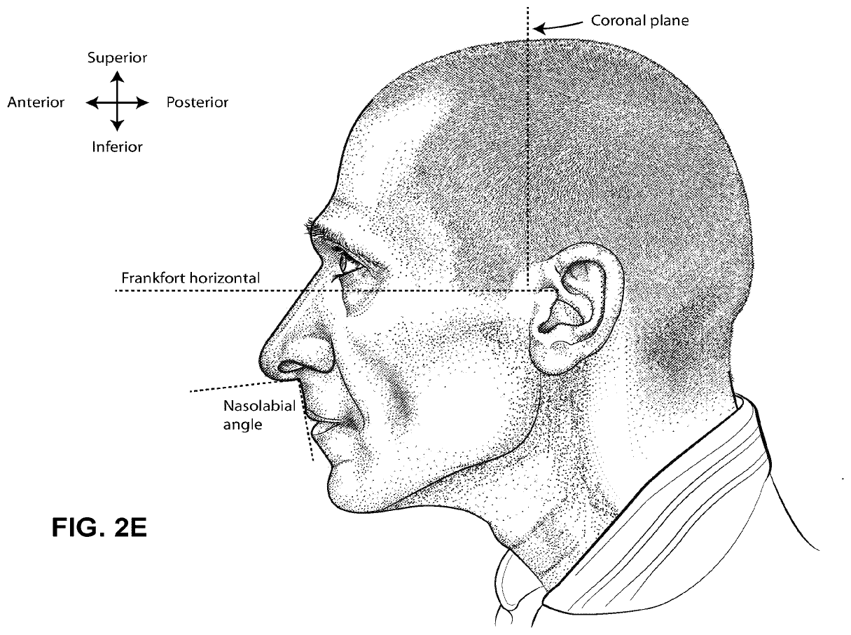

FIG. 2A shows an overview of a human respiratory system including the nasal and oral cavities, the larynx, vocal folds, oesophagus, trachea, bronchus, lung, alveolar sacs, heart and diaphragm.

FIG. 2B shows a view of a human upper airway including the nasal cavity, nasal bone, lateral nasal cartilage, greater alar cartilage, nostril, lip superior, lip inferior, larynx, hard palate, soft palate, oropharynx, tongue, epiglottis, vocal folds, oesophagus and trachea.

FIG. 2C is a front view of a face with several features of surface anatomy identified including the lip superior, upper vermilion, lower vermilion, lip inferior, mouth width, endocanthion, a nasal ala, nasolabial sulcus and cheilion. Also indicated are the directions superior, inferior, radially inward and radially outward.

FIG. 2D is a side view of a head with several features of surface anatomy identified including glabella, sellion, pronasale, subnasale, lip superior, lip inferior, supramenton, nasal ridge, alar crest point, otobasion superior and otobasion inferior. Also indicated are the directions superior & inferior, and anterior & posterior.

FIG. 2E is a further side view of a head. The approximate locations of the Frankfort horizontal and nasolabial angle are indicated. The coronal plane is also indicated.

### 4.3 Patient Interface

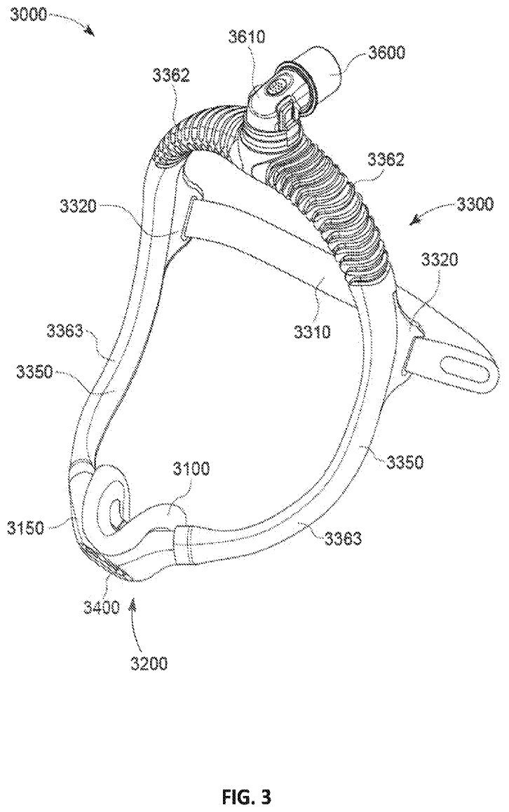

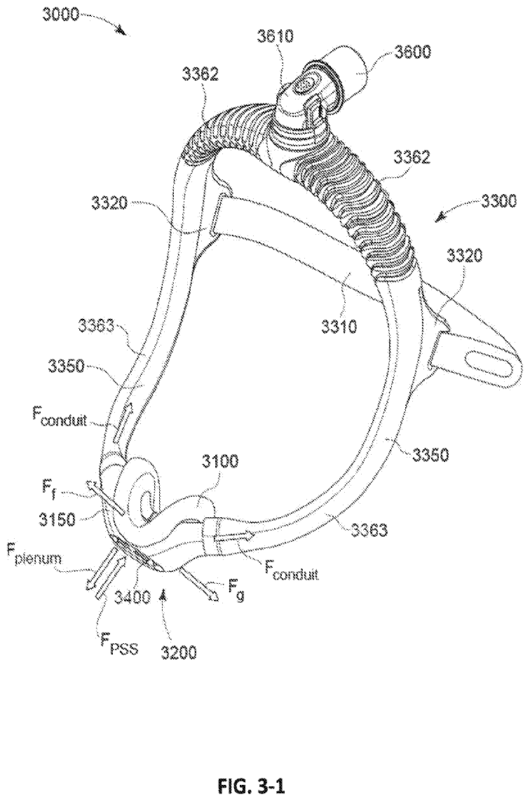

FIG. 3 shows a patient interface having conduit headgear, in accordance with one form of the present technology.

FIG. 3-1 shows forces acting on the patient interface of FIG. 3, while in use.

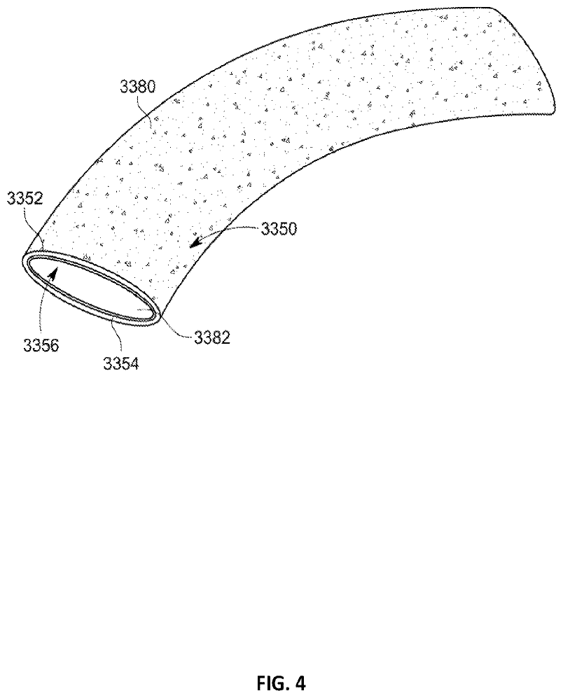

FIG. 4 shows a perspective view of a headgear tube for use in conduit headgear, in accordance with one form of the present technology.

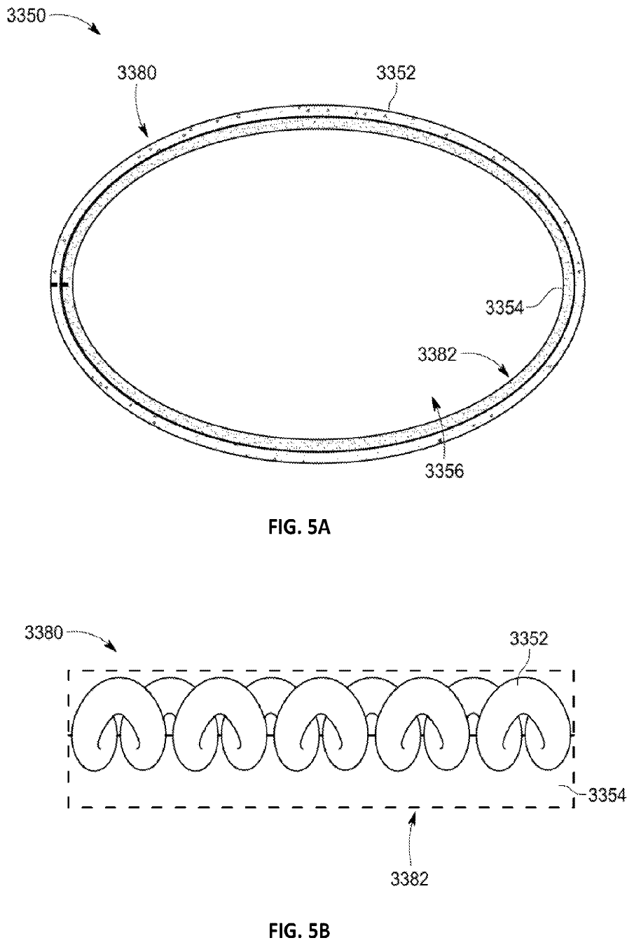

FIG. 5A shows a cross-sectional view of a headgear tube for use in conduit headgear, in accordance with one form of the present technology.

FIG. 5B shows a detail close up view of the headgear tube of FIG. 5A.

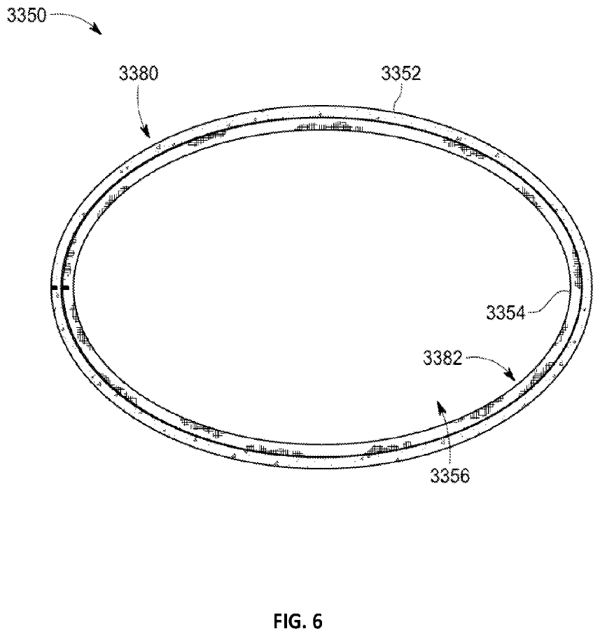

FIG. 6 shows a cross-sectional view of a headgear tube for use in conduit headgear, in accordance with one form of the present technology.

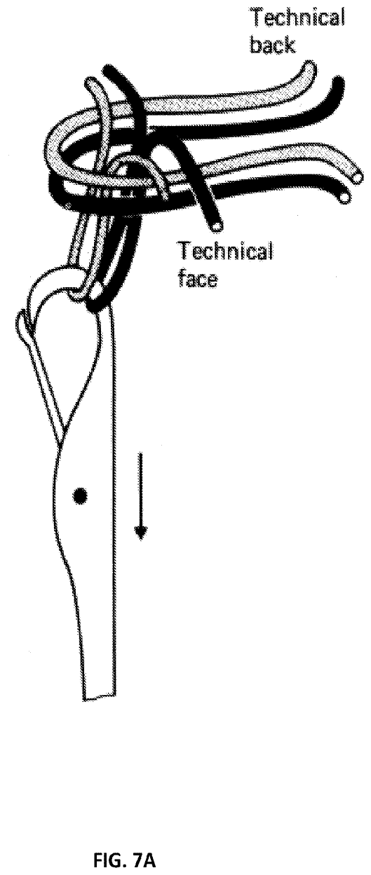

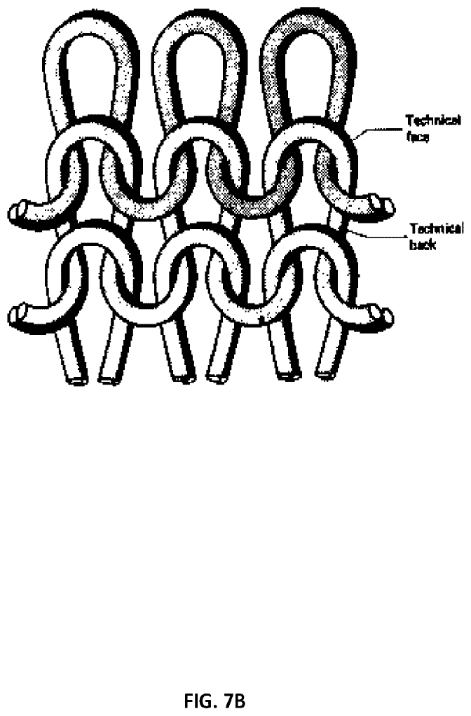

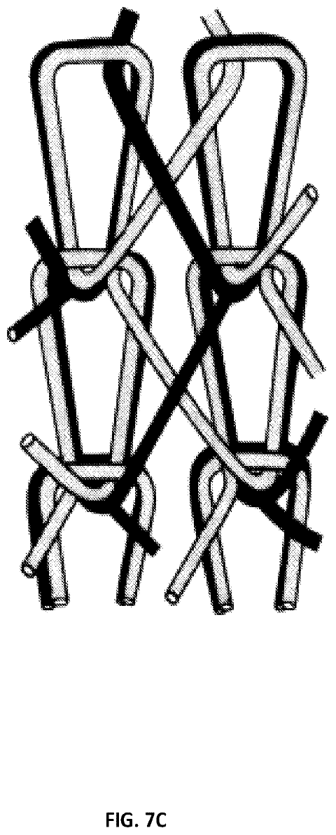

FIG. 7A shows a schematic of a plated knit structure for a headgear tube for use in conduit headgear, formed using plating techniques generally, in accordance with one form of the present technology.

FIG. 7B shows a schematic of a plated knit structure for a headgear tube for use in conduit headgear, formed using a weft knitting technique, in accordance with one form of the present technology.

FIG. 7C shows a schematic of a plated knit structure for a headgear tube for use in conduit headgear, formed using a warp knitting technique, in accordance with one form of the present technology.

### 5 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 5.1 Therapy

In one form, the present technology comprises a method for treating a respiratory disorder comprising applying positive pressure to the entrance of the airways of a patient 1000.

In certain examples of the present technology, a supply of air at positive pressure is provided to the nasal passages of the patient via one or both nares.

In certain examples of the present technology, mouth breathing is limited, restricted or prevented.

### 5.2 Respiratory Therapy Systems

In one form, the present technology comprises a respiratory therapy system for treating a respiratory disorder. The respiratory therapy system may comprise an RPT device 4000 for supplying a flow of air to the patient 1000 via an air circuit 4170 and a patient interface 3000 or 3800.

### 5.3 Patient Interface

A non-invasive patient interface 3000, such as that shown in FIG. 3A, in accordance with one aspect of the present technology comprises the following functional aspects: a seal-forming structure 3100, a plenum chamber 3200, a positioning and stabilising structure 3300, a vent 3400, one form of connection port 3600 for connection to air circuit 4170, and a forehead support 3700. In some forms a functional aspect may be provided by one or more physical components. In some forms, one physical component may provide one or more functional aspects. In use the seal-forming structure 3100 is arranged to surround an entrance to the airways of the patient so as to maintain positive pressure at the entrance(s) to the airways of the patient 1000. The sealed patient interface 3000 is therefore suitable for delivery of positive pressure therapy.

As shown in FIG. 3, a non-invasive patient interface 3000 in accordance with another aspect of the present technology comprises the following functional aspects: a seal-forming structure 3100, a plenum chamber 3200, a positioning and stabilising structure 3300, a vent 3400 and one form of connection port 3600 for connection to an air circuit (such as the air circuit 4170 shown in FIGS. 1A-1C). The plenum chamber 3200 may be formed of one or more modular components (e.g., a cushion module 3150 together with the seal-forming structure 3100) in the sense that it or they can be replaced with different components, for example components of a different size.

If a patient interface is unable to comfortably deliver a minimum level of positive pressure to the airways, the patient interface may be unsuitable for respiratory pressure therapy.

The patient interface 3000 in accordance with one form of the present technology is constructed and arranged to be able to provide a supply of air at a positive pressure above the ambient, for example at least 2, 4, 6, 10, or 20 cmH2O with respect to ambient.

### 5.3.1 Seal-Forming Structure

In one form of the present technology, a seal-forming structure 3100 provides a target seal-forming region, and may additionally provide a cushioning function. The target seal-forming region is a region on the seal-forming structure 3100 where sealing may occur. The region where sealing actually occurs—the actual sealing surface—may change within a given treatment session, from day to day, and from patient to patient, depending on a range of factors including for example, where the patient interface was placed on the face, tension in the positioning and stabilising structure and the shape of a patient's face.

In one form the target seal-forming region is located on an outside surface of the seal-forming structure 3100.

In certain forms of the present technology, the seal-forming structure 3100 is constructed from a biocompatible material, e.g. silicone rubber.

A seal-forming structure 3100 in accordance with the present technology may be constructed from a soft, flexible, resilient material such as silicone.

In certain forms of the present technology, a system is provided comprising more than one a seal-forming structure 3100, each being configured to correspond to a different size and/or shape range. For example the system may comprise one form of a seal-forming structure 3100 suitable for a large sized head, but not a small sized head and another suitable for a small sized head, but not a large sized head.

### 5.3.1.1 Sealing Mechanisms

In one form, the seal-forming structure includes a sealing flange utilizing a pressure assisted sealing mechanism. In use, the sealing flange can readily respond to a system positive pressure in the interior of the plenum chamber 3200 acting on its underside to urge it into tight sealing engagement with the face. The pressure assisted mechanism may act in conjunction with elastic tension in the positioning and stabilising structure.

In one form, the seal-forming structure 3100 comprises a sealing flange and a support flange. The sealing flange comprises a relatively thin member with a thickness of less than about 1 mm, for example about 0.25 mm to about 0.45 mm, which extends around the perimeter of the plenum chamber 3200. Support flange may be relatively thicker than the sealing flange. The support flange is disposed between the sealing flange and the marginal edge of the plenum chamber 3200, and extends at least part of the way around the perimeter. The support flange is or includes a spring-like element and functions to support the sealing flange from buckling in use.

In one form, the seal-forming structure may comprise a compression sealing portion or a gasket sealing portion. In use the compression sealing portion, or the gasket sealing portion is constructed and arranged to be in compression, e.g. as a result of elastic tension in the positioning and stabilising structure.

In one form, the seal-forming structure comprises a tension portion. In use, the tension portion is held in tension, e.g. by adjacent regions of the sealing flange.

In certain forms of the present technology, a seal-forming structure may comprise one or more of a pressure-assisted sealing flange, a compression sealing portion, a gasket sealing portion, a tension portion, and a portion having a tacky or adhesive surface.

### 5.3.1.2 Nose Bridge or Nose Ridge Region

In one form, the non-invasive patient interface 3000 comprises a seal-forming structure that forms a seal in use on a nose bridge region or on a nose-ridge region of the patient's face.

In one form, the seal-forming structure includes a saddle-shaped region constructed to form a seal in use on a nose bridge region or on a nose-ridge region of the patient's face.

### 5.3.1.3 Upper Lip Region

In one form, the non-invasive patient interface 3000 comprises a seal-forming structure that forms a seal in use on an upper lip region (that is, the lip superior) of the patient's face.

In one form, the seal-forming structure includes a saddle-shaped region constructed to form a seal in use on an upper lip region of the patient's face.

### 5.3.1.4 Chin-Region

In one form the non-invasive patient interface 3000 comprises a seal-forming structure that forms a seal in use on a chin-region of the patient's face.

In one form, the seal-forming structure includes a saddle-shaped region constructed to form a seal in use on a chin-region of the patient's face.

### 5.3.1.5 Forehead Region

In one form, the seal-forming structure that forms a seal in use on a forehead region of the patient's face. In such a form, the plenum chamber may cover the eyes in use.

### 5.3.1.6 Nasal Pillows

In one form the seal-forming structure of the non-invasive patient interface 3000 comprises a pair of nasal puffs, or nasal pillows, each nasal puff or nasal pillow being constructed and arranged to form a seal with a respective naris of the nose of a patient.

Nasal pillows in accordance with an aspect of the present technology include: a frusto-cone, at least a portion of which forms a seal on an underside of the patient's nose, a stalk, a flexible region on the underside of the frusto-cone and connecting the frusto-cone to the stalk. In addition, the structure to which the nasal pillow of the present technology is connected includes a flexible region adjacent the base of the stalk. The flexible regions can act in concert to facilitate a universal joint structure that is accommodating of relative movement both displacement and angular of the frusto-cone and the structure to which the nasal pillow is connected. For example, the frusto-cone may be axially displaced towards the structure to which the stalk is connected.

### 5.3.1.7 Nose-Only Masks

In one form, the patient interface 3000 comprises a seal-forming structure 3100 configured to seal around an entrance to the patient's nasal airways but not around the patient's mouth. The seal-forming structure 3100 may be configured to seal to the patient's lip superior. The patient interface 3000 may leave the patient's mouth uncovered. This patient interface 3000 may deliver a supply of air or breathable gas to both nares of patient 1000 and not to the mouth. This type of patient interface may be identified as a nose-only mask.

One form of nose-only mask according to the present technology is what has traditionally been identified as a “nasal mask”, having a seal-forming structure 3100 configured to seal on the patient's face around the nose and over the bridge of the nose. A nasal mask may be generally triangular in shape. In one form, the non-invasive patient interface 3000 comprises a seal-forming structure 3100 that forms a seal in use to an upper lip region (e.g. the lip superior), to the patient's nose bridge or at least a portion of the nose ridge above the pronasale, and to the patient's face on each lateral side of the patient's nose, for example proximate the patient's nasolabial sulci. The patient interface 3000 shown in FIG. 1B has this type of seal-forming structure 3100. This patient interface 3000 may deliver a supply of air or breathable gas to both nares of patient 1000 through a single orifice.

Another form of nose-only mask may seal around an inferior periphery of the patient's nose without engaging the user's nasal ridge. This type of patient interface 3000 may be identified as a “nasal cradle” mask and the seal-forming structure 3100 may be identified as a “nasal cradle cushion”, for example. In one form, for example as shown in FIG. 3, the seal-forming structure 3100 is configured to form a seal in use with inferior surfaces of the nose around the nares. The seal-forming structure 3100 may be configured to seal around the patient's nares at an inferior periphery of the patient's nose including to an inferior and/or anterior surface of a pronasale region of the patient's nose and to the patient's nasal alae. The seal-forming structure 3100 may seal to the patient's lip superior. The shape of the seal-forming structure 3100 may be configured to match or closely follow the underside of the patient's nose and may not contact a nasal bridge region of the patient's nose or any portion of the patient's nose superior to the pronasale. In one form of nasal cradle cushion, the seal-forming structure 3100 comprises a bridge portion dividing the opening into two orifices, each of which, in use, supplies air or breathable gas to a respective one of the patient's nares. The bridge portion may be configured to contact or seal against the patient's columella in use. Alternatively, the seal-forming structure 3100 may comprise a single opening to provide a flow or air or breathable gas to both of the patient's nares.

In some forms, a nose-only mask may comprise nasal pillows, described above.

### 5.3.1.8 Nose and Mouth Masks

In one form, the patient interface 3000 comprises a seal-forming structure 3100 configured to seal around an entrance to the patient's nasal airways and also around the patient's mouth. The seal-forming structure 3100 may be configured to seal to the patient's face proximate a chin region. This patient interface 3000 may deliver a supply of air or breathable gas to both nares and to the mouth of patient 1000. This type of patient interface may be identified as a nose and mouth mask.

One form of nose-and-mouth mask according to the present technology is what has traditionally been identified as a “full-face mask”, having a seal-forming structure 3100 configured to seal on the patient's face around the nose, below the mouth and over the bridge of the nose. A nose-and-mouth mask may be generally triangular in shape. In one form the patient interface 3000 comprises a seal-forming structure 3100 that forms a seal in use to a patient's chin-region (which may include the patient's lip inferior and/or a region directly inferior to the lip inferior), to the patient's nose bridge or at least a portion of the nose ridge superior to the pronasale, and to cheek regions of the patient's face. The patient interface 3000 shown in FIG. 1C is of this type. This patient interface 3000 may deliver a supply of air or breathable gas to both nares and mouth of patient 1000 through a single orifice. This type of seal-forming structure 3100 may be referred to as a “nose-and-mouth cushion”.

In another form the patient interface 3000 comprises a seal-forming structure 3100 that forms a seal in use on a patient's chin region (which may include the patient's lip inferior and/or a region directly inferior to the lip inferior), to an inferior and/or an anterior surface of a pronasale portion of the patient's nose, to the alae of the patient's nose and to the patient's face on each lateral side of the patient's nose, for example proximate the nasolabial sulci. The seal-forming structure 3100 may also form a seal against a patient's lip superior. A patient interface 3000 having this type of seal-forming structure may have a single opening configured to deliver a flow of air or breathable gas to both nares and mouth of a patient, may have an oral hole configured to provide air or breathable gas to the mouth and a nasal hole configured to provide air or breathable gas to the nares, or may have an oral hole for delivering air to the patient's mouth and two nasal holes for delivering air to respective nares. This type of patient interface 3000 may have a nasal portion and an oral portion, the nasal portion sealing to the patient's face at similar locations to a nasal cradle mask.

In a further form of nose and mouth mask, the patient interface 3000 may comprise a seal-forming structure 3100 having a nasal portion comprising nasal pillows and an oral portion configured to form a seal to the patient's face around the patient's mouth.

In some forms, the seal-forming structure 3100 may have a nasal portion that is separate and distinct from an oral portion. In other forms, a seal-forming structure 3100 may form a contiguous seal around the patient's nose and mouth.

It is to be understood that the above examples of different forms of patient interface 3000 do not constitute an exhaustive list of possible configurations. In some forms a patient interface 3000 may comprise a combination of different features of the above described examples of nose-only and nose and mouth masks.

### 5.3.2 Plenum Chamber

The plenum chamber 3200 has a perimeter that is shaped to be complementary to the surface contour of the face of an average person in the region where a seal will form in use. In use, a marginal edge of the plenum chamber 3200 is positioned in close proximity to an adjacent surface of the face. Actual contact with the face is provided by the seal-forming structure 3100. The seal-forming structure 3100 may extend in use about the entire perimeter of the plenum chamber 3200. In some forms, the plenum chamber 3200 and the seal-forming structure 3100 are formed from a single homogeneous piece of material.

In certain forms of the present technology, the plenum chamber 3200 does not cover the eyes of the patient in use. In other words, the eyes are outside the pressurised volume defined by the plenum chamber. Such forms tend to be less obtrusive and/or more comfortable for the wearer, which can improve compliance with therapy.

In certain forms of the present technology, the plenum chamber 3200 is constructed from a transparent material, e.g. a transparent polycarbonate. The use of a transparent material can reduce the obtrusiveness of the patient interface, and help improve compliance with therapy. The use of a transparent material can aid a clinician to observe how the patient interface is located and functioning.

In certain forms of the present technology, the plenum chamber 3200 is constructed from a translucent material. The use of a translucent material can reduce the obtrusiveness of the patient interface, and help improve compliance with therapy.

In some forms, the plenum chamber 3200 is constructed from a rigid material such as polycarbonate. The rigid material may provide support to the seal-forming structure.

In some forms, the plenum chamber 3200 is constructed from a flexible material (e.g., constructed from a soft, flexible, resilient material like silicone, textile, foam, etc.). For example, in examples then may be formed from a material which has a Young's modulus of 0.4 GPa or lower, for example foam. In some forms of the technology the plenum chamber 3200 may be made from a material having Young's modulus of 0.1 GPa or lower, for example rubber. In other forms of the technology the plenum chamber 3200 may be made from a material having a Young's modulus of 0.7 MPa or less, for example between 0.7 MPa and 0.3 MPa. An example of such a material is silicone.

### 5.3.3 Positioning and Stabilising Structure

The seal-forming structure 3100 of the patient interface 3000 of the present technology may be held in sealing position in use by the positioning and stabilising structure 3300. The positioning and stabilising structure 3300 may comprise and function as “headgear” since it engages the patient's head in order to hold the patient interface 3000 in a sealing position. An example of a positioning and stabilising structure may be shown in FIG. 3.

In one form the positioning and stabilising structure 3300 provides a retention force at least sufficient to overcome the effect of the positive pressure in the plenum chamber 3200 to lift off the face (i.e., Fplenum).

In one form the positioning and stabilising structure 3300 provides a retention force to overcome the effect of the gravitational force on the patient interface 3000.

In one form the positioning and stabilising structure 3300 provides a retention force as a safety margin to overcome the potential effect of disrupting forces on the patient interface 3000, such as from tube drag, or accidental interference with the patient interface.

In one form of the present technology, a positioning and stabilising structure 3300 is provided that is configured in a manner consistent with being worn by a patient while sleeping. In one example the positioning and stabilising structure 3300 has a low profile, or cross-sectional thickness, to reduce the perceived or actual bulk of the apparatus. In one example, the positioning and stabilising structure 3300 comprises at least one strap having a rectangular cross-section. In one example the positioning and stabilising structure 3300 comprises at least one flat strap.

### 5.3.3.1 Conduit Headgear

### 5.3.3.1.1 Conduit Headgear Tubes

In some forms of the present technology, the positioning and stabilising structure 3300 comprises one or more headgear or conduit tubes 3350 that deliver pressurised air received from a conduit forming part of the air circuit 4170 from the RPT device to the patient's airways, for example through the plenum chamber 3200 and seal-forming structure 3100. In the form of the present technology illustrated in FIG. 3, the positioning and stabilising structure 3300 comprises two headgear or conduit tubes 3350 that deliver air to the plenum chamber 3200 from the air circuit 4170. This type of patient interface 3000 is an arrangement may be referred to as conduit headgear.

The tubes 3350 are configured to position and stabilise the seal-forming structure 3100 of the patient interface 3000 at the appropriate part of the patient's face (for example, the nose and/or mouth) in use. This allows the conduit of air circuit 4170 providing the flow of pressurised air to connect to a connection port 3600 of the patient interface in a position other than in front of the patient's face, for example on top of the patient's head.

In the form of the present technology illustrated in FIG. 3, the positioning and stabilising structure 3300 comprises two tubes 3350, each tube 3350 being positioned in use on a different side of the patient's head and extending across the respective cheek region, above the respective ear (superior to the otobasion superior on the patient's head) to the elbow 3610 on top of the head of the patient 1000. This form of technology may be advantageous because, if a patient sleeps with their head on its side and one of the tubes 3350 is compressed to block or partially block the flow of gas along the tube 3350, the other tube 3350 remains open to supply pressurised gas to the patient. In other examples of the technology, the patient interface 3000 may comprise a different number of tubes, for example one tube, or two or more tubes.

In one example in which the patient interface has one tube 3350, the single tube 3350 is positioned on one side of the patient's head in use (e.g. across one cheek region) and a strap forms part of the positioning and stabilising structure 3300 and is positioned on the other side of the patient's head in use (e.g. across the other region) to assist in securing the patient interface 3000 on the patient's head. For example, the tube 3350 and the strap may each be under tension in use in order to assist in maintaining the seal-forming structure 3100 in a sealing position.

In one form, the tube 3350 may be at least partially extensible so that the tube 3350 and the strap may adjust substantially equal lengths when worn by a patient. This may allow for substantially symmetrical adjustments between the tube 3350 and the strap so that the seal-forming structure remains substantially in the middle.

In the form of the technology shown in FIG. 3, the two tubes 3350 are fluidly connected at superior ends to each other and to the connection port 3600. In some examples, the two tubes 3350 are integrally formed while in other examples the tubes 3350 are formed separately but are connected in use and may be disconnected, for example for cleaning or storage. Where separate tubes are used, they may be indirectly connected together, for example each may be connected to a T-shaped connector. The T-shaped connector may have two arms/branches each fluidly connectable to a respective one of the tubes 3350. Additionally, the T-shaped connector may have a third arm or opening providing the connection port 3600 for fluid connection to the air circuit 4170 in use.

The tubes 3350 may be formed from a flexible material, such as an elastomer, e.g. silicone or TPE, such as is shown in FIG. 3. However, the tubes may also or instead be formed from one or more textile and/or foam materials.

The tubes 3350 may have a preformed shape and may be able to be bent or moved into another shape upon application of a force but may return to the original preformed shape in the absence of said force. The tubes 3350 may be generally arcuate or curved in a shape approximating the contours of a patient's head between the top of the head and the nasal or oral region.

In some examples, the one or more tubes 3350 are crush resistant to resist being blocked if crushed during use, for example if squashed between a patient's head and pillow, especially if there is only one tube 3350. The tubes 3350 may be formed with a sufficient structural stiffness to resist crushing or may be as described in U.S. Pat. No. 6,044,844, the contents of which are incorporated herein by reference.

Each tube 3350 may be configured to receive a flow of air from the connection port 3600 on top of the patient's head and to deliver the flow of air to the seal-forming structure 3100 at the entrance of the patient's airways. In the example shown in FIG. 3, each tube 3350 lies in use on a path extending from the plenum chamber 3200 across the patient's cheek region and superior to the patient's ear to the elbow 3610. For example, a portion of each tube 3350 proximate the plenum chamber 3200 may overlie a maxilla region of the patient's head in use. Another portion of each tube 3350 may overlie a region of the patient's head superior to an otobasion superior of the patient's head. Each of the tubes 3350 may also lie over the patient's sphenoid bone and/or temporal bone and either or both of the patient's frontal bone and parietal bone. The elbow 3610 may be located in use over the patient's parietal bone, over the frontal bone and/or over the junction therebetween (e.g. the coronal suture).

In certain forms of the present technology the patient interface 3000 is configured such that the connection port 3600 can be positioned in a range of positions across the top of the patient's head so that the patient interface 3000 can be positioned as appropriate for the comfort or fit of an individual patient. In some examples, the headgear tubes 3350 are configured to allow movement of an upper portion of the patient interface 3000 (e.g. a connection port 3600) with respect to a lower portion of the patient interface 3000 (e.g. a plenum chamber 3200). That is, the connection port 3600 may be at least partially decoupled from the plenum chamber 3200. In this way, the seal-forming structure 3100 may form an effective seal with the patient's face irrespective of the position of the connection port 3600 (at least within a predetermined range of positions) on the patient's head.

As described above, in some examples of the present technology the patient interface 3000 comprises a seal-forming structure 3100 in the form of a cradle cushion which lies generally under the nose and seals to an inferior periphery of the nose (e.g. an under-the-nose cushion). The positioning and stabilising structure 3300, including the tubes 3350 may be structured and arranged to pull the seal-forming structure 3100 into the patient's face under the nose with a sealing force in a posterior and superior direction (e.g. a posterosuperior direction). A sealing force with a posterosuperior direction may cause the seal-forming structure 3100 to form a good seal to both the inferior periphery of the patient's nose and anterior-facing surfaces of the patient's face, for example on either side of the patient's nose and the patient's lip superior.

Conduits forming part of the positioning and stabilising structure 3300, like headgear straps, may provide a force that contributes to the positioning and stabilising force FPSS. As illustrated in FIG. 3-1, the positioning and stabilising force FPSS may be the resultant force from the various forces of the different elements of the positioning and stabilising structure 3300. For example, each conduit may provide a force Fconduit directed in the posterior and respective lateral direction in order to hold the seal-forming structure 3100 against the patient's face (into the upper lip and sealing under the nose) and oppose the effect of the positive pressure in the plenum chamber 3200 to lift off the face (i.e., Fplenum). The force Fconduit directed may also be directed at least partially in the superior direction in order to overcome the gravitational force Fg.

In some forms, the conduits may provide a force directed into the patient's head when the conduits are filled with pressurized air. The force may assist in gripping the patient's head. The force may be caused by the inflation of the conduits during normal use. In some forms, the force may provide a cushioning effect to the patient's head. The conduits may be designed in order to limit expansion in order to prevent over-gripping the patient's head.

The position of the patient's head may also change the gripping force of the conduits. For example, if the patient is sleeping on his side, the weight of the patient's head may compress one conduit, and the other conduit (e.g., the lateral portion not between the patient's head and a sleeping surface, like a pillow) may additionally expand in order to keep substantially the same flow rate of pressurized air.

The gravitational force Fg may be opposed by a frictional force Ff, which may act in a direction directly opposite of the gravitational force Fg. As gravity pulls the seal-forming structure 3100 and the plenum chamber 3200 in the inferior direction, the frictional force Ff would act in the superior direction (e.g., against a patient's face). For example, the patient may experience the frictional force Ff against his lip superior (and/or other surfaces of the patient's face in contact with the seal-forming structure 3100) in order to oppose the motion in the inferior direction (which may help to stabilising the cushion in place). Although the frictional force Ff is shown specifically opposing the gravitational force Fg of the seal-forming structure 3100 and the plenum chamber 3200, components of an overall frictional force (not shown) would also oppose the gravitational force Fg associated with the positioning and stabilising structure 3300 and any other portions of the patient interface 3000. A force of friction can act along any place where the patient interface 3000 contacts the patient's skin (or hair). The frictional force Ff extends in the opposite direction of the gravitational force Fg and along the patient's skin (or hair).

In some forms, the sum of the various forces may equal zero so that the patient interface 3000 is at equilibrium (e.g., not moving along the patient's face while in use). Specifically, the gravitational force Fg and the blowout force Fplenum tend to move the seal-forming structure 3100 away from the desired sealing position. The positioning and stabilising force FPSS is applied in order to counteract the gravitational force Fg and the blowout force Fplenum (as well as any frictional forces Ff) and keep the seal-forming structure 3100 properly situated. Although the positioning and stabilising force FPSS may exceed the sum of the gravitational force Fg and the blowout force Fplenum (with any additional positioning and stabilising force FPSS being balanced by reaction force from the patient's head acting on the portions of patient interface 3000) and still maintain the seal-forming structure 3100 in an appropriate sealing position, patient comfort may be sacrificed. Maximum patient comfort may be achieved when the net force on the patient interface 3000 is zero and the positioning and stabilising force FPSS is exactly strong enough to achieve this. In some examples the positioning and stabilising structure 3300 may be adjustable such that when fitted the positioning and stabilising force FPSS is greater than required to exactly balance the gravitational force Fg and the blowout force Fplenum to hold the patient interface 3000 against the patient's head tightly enough that disruptive forces which may be experienced in use (such as tube drag or lateral shunting of the plenum chamber 3200 during side sleeping) do not disrupt the seal. As described below, various positions of the patient's head while using the patient interface 3000 may determine the positioning and stabilising force FPSS necessary to achieve equilibrium.

### 5.3.3.1.2 Textile Headgear Tube

As noted above, the headgear or conduit tube 3350 may be comprised, at least partially, of a textile. An example of this is shown in FIG. 4, which depicts a tube 3350 in a perspective view. It can be seen that it comprises an outer textile layer 3352, which forms the outer surface 3380, i.e. the patient contacting side of the tube, and a second impermeable layer 3354 defining the inner side 3382 of the tube, the interior 3356 of the tube forming a flow path for air moving therethrough. Providing at least the outer surface of the tube with a textile material may provide a softer and more comfortable experience for the patient wearing the conduit headgear incorporating the tube. The tube may be formed from textile materials in a number of ways, and examples of these follow.

In one example shown in FIG. 5A, the tube 3350 has been formed as a plated knit structure. A plated knit structure contains loops composed of two or more lengths of yarn which are supplied separately to the same needle hook through their own guides. These guides influence the respective position of the lengths of yarn relative to the respective sides of the plated knit structure. Although the respective yarns become linked or otherwise intermeshed with each other when forming the textile, one yarn becomes more prominent or dominant on one side and the other yarn becomes more prominent on the other side due to how they overlap each other. In general, the length of yarn positioned closest to the needle head during manufacture of the textile is more prominent on the technical back of the textile layer and while the other yarn is lower, relative to the first yarn, and shows on the technical face of the textile. It will be understood that in this example, the technical face is the outer surface of the tube 3350 and the technical back is the inner surface of the tube.

FIGS. 7A to 7C show examples of how the technical face and technical backs may be formed using plating techniques generally (FIG. 7A), weft knitting techniques (FIG. 7B) and warp knitting (FIG. 7C). These techniques allow for a textile which combines different yarns used for the technical face and technical back, thereby allowing for these surfaces to be selected to maximise patient comfort and/or airtightness. In other words, the first yarn may be knitted to the second yarn to form a wall of a conduit where the wall defines a hollow interior through which a flow of air passes in use.

The use of a plated knit structure for the tube 3350 is advantageous since it allows the tube to be formed as a continuous structure, without any obvious seams, either lengthwise along the headgear tube or circumferentially about the tube that may cause discomfort for the patient if these structures bear against the skin.

In forming the illustrated example of the tube 3350, the respective lengths of yarn have been selected for their specific physical properties. The yarn selected for technical face forms the outer surface 3380 of the headgear tube, and in some examples is a soft and breathable yarn, such as a synthetic and/or natural yarn or a blend of same. This is preferred for patient comfort when the headgear tube contacts the skin. In non-limiting examples, the yarn may comprise polyamide material. In one such non-limiting example, the technical face comprise of a yarn of nylon. In another non-limiting example, the yarn of the first textile layer may comprise polyurethane material. In one such non-limiting example, the first layer may comprise a yarn of polyurethane co-polymer material such as elastane (e.g. spandex, Lycra®, or ROICA™) or the like. However, yarns of other materials may be selected depending on the desired properties; for example, the yarn may be selected for a preferred aesthetic appearance such as its colour and bulk. In some instances, the yarn may be selected for a particular cross-section profile, which could affect its ability to retain dyes and reflect light, among other factors.

In this example, the yarn selected for the technical back is a phase changing polymer yarn, i.e. when activated it changes from a first phase to a second phase. In non-limiting examples, the yarn of the technical back is one of polypropylene, polyethylene, thermoplastic polyurethane, and a thermoplastic elastomer, or a blend thereof. Other polymer yarns may be selected for their phase-changing properties. Other desired properties for the yarn of the technical back, such as colour, melting points, stiffness, tensile modulus and flexural modulus, among other factors may have a bearing on the yarn used. It will be appreciated that in use, the technical back forms the impermeable layer of the tube. This example of tube construction is useful, in that it provides options for the material from which the impermeable layer may be formed. Thus, the material for the yarn of the second textile layer may be selected depending on available materials and costs, while still being biocompatible and easy to clean once the tube has been formed.

In examples, the respective yarns are knitted together by a needle to form the plated knit structure. As it is the technical face that, in use, contacts the patient's skin, the use of a softer yarn for this surface may make for a more comfortable experience when the conduit headgear is being worn.

In some examples the conduit may be formed using a first textile layer 3352 and a second textile layer 3354 which may be combined using knitting or any other process known to those skilled in the art. When combined the first textile layer 3352 may form the outer, or patient contacting side of the conduit, and the second textile layer 3354 may form the inner, air impermeable side of the conduit. It should be appreciated that the result of both approaches, i.e., combining yarns to form a textile conduit, or combining textile layers to form a textile conduit result in a conduit having a wall which defines a hollow interior through which the flow of air passes in use, the wall having the desired properties of a soft outer surface and a substantially air impermeable inner surface. As such for sake of efficiency, reference is made herein to first 3352 and second textile layers 3354, however these can interchangeably refer to yarns selected for the technical back and technical face without departing from the scope of the present invention.

Once the plated knit structure has been formed the yarn comprising the second textile layer/technical back is activated such that it undergoes a phase change. This causes the yarn to become molten such that it coalesces with the first textile layer. The plate knit structure may then be allowed to cool, or is actively cooled, such that the yarn becomes solid, forming an inner surface that is continuous and substantially air impermeable along the interior of the tube. This technique is advantageous as it provides the opportunity to form the tube in the desired shape for use with the conduit headgear, which may need to conform somewhat to the shape of the patient's head. Additionally, forming the conduit tube from a plated knit structure such as herein described may provide a reduction in the weight of patient interfaces using conduit headgear, due to the absence of silicone tubes. It also provides a conduit tube with a substantially unitary structure, where the exterior layer is not able to move relative to the interior layer, potentially reducing snagging resistance as the conduit headgear moves across bedding or such like.

The interaction of the respective first 3352 and second textile layers 3354 of the finished tube is best seen in FIG. 5B; this shows a close up detail of the cross-section of the tube shown in FIG. 5A. In this view, the first textile layer is seen as being comprised of a series of whorls and/or loops. This provides a soft surface for the exterior surface 3380, or technical face, of the headgear tube. Having been activated such that the yarn forming the second textile layer becomes molten, it has permeated and flows or otherwise meanders through at least a portion of the whorls and loops of the first textile layer. When the yarn of the second textile layer sets, through the use of a setting or cooling agent or such like, it hardens and bonds with the first textile layer. As a portion of the second textile layer is now interspersed with at least a portion of the first textile layer, this effectively forms an integrated and unitary structure that forms the impermeable inner surface 3382. This provides a relatively tight, firm, and lightweight construction for the tube, with a substantially continuous and smooth inner surface. The interior of the tube may be easily cleaned by the patient as necessary to ensure appropriate hygiene is maintained.

The relative thickness of the impermeable inner surface 3382 contributes to the overall rigidity of the tube 3350 such that it can hold its shape, but is still able to deform, in response to changes in forces applied by the patient or changes in air pressure as it flows through the tube.

The phase change may be initiated in a number of ways. In one example, it may involve subjecting the plated knit structure to heat; this may be applied directly, by way of placing the plated knit structure over a mandrel or similar shaped component, such that the technical back contacts the mandrel. The mandrel may then be heated such that the second textile layer becomes molten and coalesces with the first textile layer. The plated knit structure may then be cooled, either by reducing the temperature applied to the mandrel, simply allowing the plated knit structure to cool on the mandrel or removing the plated knit structure altogether for cooling.

In another example, the phase change may be initiated through application of a chemical substance, either being sprayed directly onto the technical back or by being a coating applied to a mandrel or similar shaped component, over which the plated knit structure is positioned. In this example, the chemical may be selected for biocompatibility.

In yet another example, the yarn of the second textile layer may have been selected for its photochemical properties. When exposed to light of a certain wavelength, this may initiate the desired phase change.

This tube 3350 configuration may be less costly in manufacture compared to more conventional tubes since it provides a more integrated structure, with inner and outer layers securely bonded to each other. This construction may also be less prone to snagging since the outer textile layer does not independently move relative to the inner, impermeable layer. There is also a reduction in components due to the absence of a separate silicone tube to which a textile layer or sleeve needs to be added. As such, the manufacture of the tube 3350 may be more cost effective and may also have a reduced production time. The tube is also reduced in weight relative to one incorporating a separate silicone tube.

An alternative example of a tube 3350 having an outer textile layer 3352 and an impermeable inner layer 3354 is shown in FIG. 6. In this example, the impermeable inner layer has been formed from a silicone or polymer comprised of a material having phase changing properties.

The yarn selected for the outer textile layer 3352 may be a synthetic yarn or natural yarn, or a blend thereof. In a non-limiting example, the yarn may be comprised of a polyamide material such as a yarn of nylon. However, the yarn may alternatively be a polyurethane material or a yarn of polyurethane co-polymer material such as elastane (e.g. spandex, Lycra®, or ROICA™) or the like. Alternatively, other yarns may be selected for the outer textile layer depending on the desired physical properties, such as its colour and bulk. In some instances, the yarn may be selected for a particular cross-section profile, which could affect its ability to retain dyes and reflect light, among other factors.

The outer textile layer 3352 is formed as a sleeve having two faces, an exterior face and an interior face. In use, the exterior face corresponds to the outer surface 3380 while the interior face receives the layer of polymer 3354.

In one example, the outer textile layer 3352 may be formed as a knit structure, for example a plated knit structure but alternatively a circular or warp knit structure, combining two or more of the yarns referred to above. In this example, the technical face corresponds to the outer surface. The use of a knit structure may allow for the selection of a combination of high/low cost yarn for manufacturing and inventory efficiency. It may also provide for the possibility of increased thickness in certain regions of the tube 3350, which may be of assistance in providing a cushioning effect for the areas of the patient's face that may underly the conduit tube when the conduit headgear is worn. For example, the portion of the conduit tube corresponding, in use, to the patient's zygomatic bones (cheekbones) may be knitted to be thicker than elsewhere along the length of the tube. This may improve comfort. This structure may also provide the possibility of configuring the tube with greater or lesser rigidity along its length as desired, for conformity to the patient's head and/or resisting deformation forces from certain directions. It will be appreciated that the outer textile layer may alternatively be formed from one or more yarns using braiding, weaving, or even sewing techniques.

The impermeable layer 3354 may be comprised of a phase changing material. In one non-limiting example, the impermeable layer may be an elastomer such as silicone rubber of an appropriate biomedical grade. Alternatively, the impermeable layer may be comprised of one or more of a polyurethane (PU), a thermoplastic polyurethane (TPU), a thermoplastic elastomer (TPE), a biodegradable thermoplastic polyurethane (bTPU). In yet another example, the impermeable layer may be a silane-polyisobutylene (Si-PIB) with one or more of the preceding polymers. Another alternative material may be a low melt yarn which may be beneficial in conferring structural integrity and strength to the outer textile layer.

Other suitable thermoplastics, thermosets and elastomers may be used, depending on the desired properties, which may include colour and melting points among others.

The impermeable layer 3354 may be applied to the inner surface of the outer textile layer 3352 in a number of ways. In one example, the impermeable layer may be applied as an aerosol coating of liquid. In another example, it may be applied as a film or web. In yet a further example, it may be applied as a liquid dip or coating.

Once applied to the outer textile layer 3352, the impermeable layer 3354 undergoes a phase change. This may involve heating the tube 3350 comprising the combined outer textile layer and impermeable layer in, or on, a suitable apparatus, such as an oven, mandrel or drying trays or the like. This results in the impermeable layer coalescing, permeating or otherwise bonding with the loops of yarn on the inner face of the first textile layer. The tube may then be removed or otherwise cooled such that the impermeable layer sets, forming the interior surface 3382 of the tube.

In some instances, the techniques required for application of the layer of polymer 3354 may require inversion of the outer textile layer 3352, i.e. the outer textile layer is turned inside out. It will be understood that this places what would otherwise be the inner surface of the outer textile layer exposed for easy application of the impermeable layer. In these instances, once the impermeable layer has undergone its phase change, the outer textile layer may be reverted such that the outer surface 3380 of the tube 3350 is exposed.

In some instances, an additional polymer may be used as a bonding agent. In this example, the additional polymer may be applied to the inner surface of the outer textile layer prior to the application of the impermeable layer.

This arrangement is useful since, like the previous example described, it does away with the need to form the impermeable layer, e.g. the silicone tube of the prior art, first before adding a textile layer about the outer surfaces of same. The tube of the present technology is an integrated, unitary structure, having fewer components relative to conventional tubes, while still being lightweight, has some ability to maintain a desired shape and retain some flexibility.

### 5.3.3.1.3 Extendable and Non-Extendable Tube Portions

In some examples of the present technology, one or both of the tubes 3350 are not extendable in length. However, in some forms, the tubes 3350 may comprise one or more extendable tube sections, for example formed by an extendable concertina structure. In some forms, the patient interface 3000 may comprise a positioning and stabilising structure 3300 including at least one gas delivery tube comprising a tube wall having an extendable concertina structure. The patient interface 3000 shown in FIG. 3 comprises tubes 3350, the superior portions of which comprise extendable tube sections each in the form of an extendable concertina structure 3362.

In some forms, the extendable concertina structure 3328 may be formed as a series of ridges and grooves on the surface of the tubes 3350. The concertina structure 3328 may be biased toward a retracted position, and may move to an expanded position when the patient dons the positioning and stabilising structure 3300. Because portions of the tubes 3350 may be substantially inextensible (e.g., non-extendable tube sections 3363), the concertina structures 3328 permit the positioning and stabilising structure 3300 to stretch in order to fit different sized heads. This may allow a single sized tube 3350 to be used with multiple sized heads. For example, the positioning and stabilising structure 3300 may be “one-size-fits-all” as a result of the concertina structure 3328. Alternatively, the tubes 3350 may be manufactured in multiple sizes (e.g., small, medium, large). The patient may select a length that most closely conforms to their head, and the concertina structures 3328 may make small adjustments in order to tailor the fit to the individual patient.

The cross-sectional shape of the non-extendable tube sections 3363 of the tubes 3350 may be circular, elliptical, oval, D-shaped or a rounded rectangle, for example as described in U.S. Pat. No. 6,044,844. A cross-sectional shape that presents a flattened surface of tube on the side that faces and contacts the patient's face or other part of the head may be more comfortable to wear than, for example a tube with a circular cross-section.

In some examples of the present technology, the non-extendable tube sections 3363 connects to the plenum chamber 3200 from a low angle. The headgear tubes 3350 may extend inferiorly down the sides of the patient's head and then curve anteriorly and medially to connect to the plenum chamber 3200 in front of the patient's face. The tubes 3350, before connecting to the plenum chamber 3200, may extend to a location at the same vertical position as (or, in some examples, inferior to) the connection with the plenum chamber 3200. That is, the tubes 3350 may project in an at least partially superior direction before connecting with the plenum chamber 3200. A portion of the tubes 3350 may be located inferior to the plenum chamber 3200 and/or the seal forming structure 3100. The tubes 3350 may contact the patient's face below the patient's cheekbones, which may be more comfortable than contact on the patient's cheekbones and may avoid excessively obscuring the patient's peripheral vision.

### 5.3.3.1.4 Conduit Headgear Connection Port

In certain forms of the present technology, the patient interface 3000 may comprise a connection port 3600 located proximal to a superior, lateral or posterior portion of a patient's head. For example, in the form of the present technology illustrated in FIG. 3, the connection port 3600 is located on top of the patient's head (e.g. at a superior location with respect to the patient's head). In this example the patient interface 3000 comprises an elbow 3610 forming the connection port 3600. The elbow 3610 may be configured to fluidly connect with a conduit of an air circuit 4170. The elbow 3610 may be configured to swivel with respect to the positioning and stabilising structure 3300 to at least partially decouple the conduit from the positioning and stabilising structure 3300. In some examples the elbow 3610 may be configured to swivel by rotation about a substantially vertical axis and, in some particular examples, by rotation about two or more axes. In some examples the elbow may comprise or be connected to the tubes 3350 by a ball-and-socket joint. The connection portion 3600 may be located in the sagittal plane of the patient's head in use.

Patient interfaces having a connection port that is not positioned anterior to the patient's face may be advantageous as some patients may find a conduit that connects to a patient interface anterior to their face to be unsightly and/or obtrusive. For example, a conduit connecting to a patient interface anterior to the patient's face may be prone to interference with bedclothes or bed linen, particularly if the conduit extends inferiorly from the patient interface in use. Forms of the present technology comprising a patient interface having a connection port positioned superiorly to the patient's head in use may make it easier or more comfortable for a patient to lie or sleep in one or more of the following positions: a side-sleeping position, a supine position (e.g. on their back, facing generally upwards) or in a prone position (e.g. on their front, facing generally downwards). Moreover, connecting a conduit to an anterior portion of a patient interface may exacerbate a problem known as tube drag in which the conduit exerts an undesired force upon the patient interface during movement of the patient's head or the conduit, thereby causing dislodgement away from the face. Tube drag may be less of a problem when force is received at a superior location of the patient's head than anterior to the patient's face proximate to the seal-forming structure (where tube drag forces may be more likely to disrupt the seal).

### 5.3.3.1.5 Headgear Tube Fluid Connections

The two tubes 3350 are fluidly connected at their inferior ends to the plenum chamber 3200. In certain forms of the technology, the connection between the tubes 3350 and the plenum chamber 3200 is achieved by connection of two rigid connectors. The tubes 3350 and plenum chamber 3200 may be configured to enable the patient to easily connect the two components together in a reliable manner. The tubes 3350 and plenum chamber 3200 may be configured to provide tactile and/or audible feedback in the form of a ‘re-assuring click’ or a similar sound, so that the patient may easily know that each tube 3350 has been correctly connected to the plenum chamber 3200. In one form, the tubes 3350 are formed from a silicone or textile material and the inferior end of each of the silicone tubes 3350 is overmolded to a rigid connector made, for example, from polypropylene, polycarbonate, nylon or the like. The rigid connector on each tube 3350 may comprise a female mating feature configured to connect with a male mating feature on the plenum chamber 3200. Alternatively, the rigid connector on each tube 3350 may comprise a male mating feature configured to connect to a female mating feature on the plenum chamber 3200. In other examples the tubes 3350 may each comprise a male or female connector formed from a flexible material, such as silicone or TPE, for example the same material from which the tubes 3350 are formed.

In other examples a compression seal is used to connect each tube 3350 to the plenum chamber 3200. For example, a resiliently flexible (e.g. silicone) tube 3350 without a rigid connector may be configured to be squeezed to reduce its diameter so that it can be compressed into a port in the plenum chamber 3200 and the inherent resilience of the silicone pushes the tube 3350 outwards to seal the tube 3350 in the port in an air-tight manner. Alternatively, in a hard-to-hard type engagement between the tube 3350 and the plenum chamber 3200, each tube 3350 and/or plenum chamber 3200 may comprise a pressure activated seal, for example a peripheral sealing flange. When pressurised gas is supplied through the tubes 3350 the sealing flange may be urged against the join between the tubes and a circumferential surface around a port or connector of the plenum chamber 3200 to form or enhance a seal between the tube 3350 and plenum chamber 3200.

### 5.3.3.2 Headgear Straps

In some forms, the positioning and stabilising structure 3300 may include at least one strap 3310 which may be worn by the patient in order to assist in properly orienting the seal-forming structure 3100 against the patient's face (e.g., in order to limit or prevent leaks). In certain forms, the strap 3310 may be at least partially extensible. For example, the strap 3310 may include elastic, or a similar extensible material.

In the example shown in FIG. 3, strap 3310 of the positioning and stabilising structure 3300 is connected between the two tubes 3350 positioned on each side of the patient's head and passing around the back of the patient's head, for example overlying or lying inferior to the occipital bone of the patient's head in use. The strap 3310 connects to each tube above the patient's ears. With reference to FIG. 3, the positioning and stabilising structure 3300 comprises a pair of tabs 3320. In use the strap 3310 may be connected between the tabs 3320. The strap 3310 may be sufficiently flexible to pass around the back of the patient's head and lie comfortably against the patient's head, even when under tension in use.

### 5.3.4 Vent

In one form, the patient interface 3000 includes a vent 3400 constructed and arranged to allow for the washout of exhaled gases, e.g. carbon dioxide.

In certain forms the vent 3400 is configured to allow a continuous vent flow from an interior of the plenum chamber 3200 to ambient whilst the pressure within the plenum chamber is positive with respect to ambient. The vent 3400 is configured such that the vent flow rate has a magnitude sufficient to reduce rebreathing of exhaled CO2 by the patient while maintaining the therapeutic pressure in the plenum chamber in use.

One form of vent 3400 in accordance with the present technology comprises a plurality of holes, for example, about 20 to about 80 holes, or about 40 to about 60 holes, or about 45 to about 55 holes.

The vent 3400 may be located in the plenum chamber 3200. Alternatively, the vent 3400 is located in a decoupling structure, e.g., a swivel.

### 5.3.5 Decoupling Structure(s)

In one form the patient interface 3000 includes at least one decoupling structure, for example, a swivel or a ball and socket.

### 5.3.6 Connection Port

Connection port 3600 allows for connection to the air circuit 4170.

### 5.3.7 Anti-Asphyxia Valve

In one form, the patient interface 3000 includes an anti-asphyxia valve.

### 5.3.8 Ports

In one form of the present technology, a patient interface 3000 includes one or more ports that allow access to the volume within the plenum chamber 3200. In one form this allows a clinician to supply supplementary oxygen. In one form, this allows for the direct measurement of a property of gases within the plenum chamber 3200, such as the pressure.

### 5.4 RPT Device

An RPT device 4000 in accordance with one aspect of the present technology comprises mechanical, pneumatic, and/or electrical components and is configured to execute one or more algorithms 4300, such as any of the methods, in whole or in part, described herein. The RPT device 4000 may be configured to generate a flow of air for delivery to a patient's airways, such as to treat one or more of the respiratory conditions described elsewhere in the present document.

### 5.5 Circuit

An air circuit 4170 in accordance with an aspect of the present technology is a conduit or a tube constructed and arranged to allow, in use, a flow of air to travel between two components such as RPT device 4000 and the patient interface 3000 or 3800.

In some examples, the air circuit may comprise one or more tubes constructed in a manner similar to that described above in respect of tubes for conduit headgear; i.e. the tubes are comprised of an outer textile layer and an inner impermeable layer, the latter having undergone a phase change during production of the tube of the air circuit. Such an air circuit may be relatively lightweight compared to air circuits that utilise silicone tubes. This may aid in patient comfort in use.

### 5.6 Glossary

For the purposes of the present technology disclosure, in certain forms of the present technology, one or more of the following definitions may apply. In other forms of the present technology, alternative definitions may apply.

### 5.6.1 General

Air: In certain forms of the present technology, air may be taken to mean atmospheric air, and in other forms of the present technology air may be taken to mean some other combination of breathable gases, e.g. oxygen enriched air.

Ambient: In certain forms of the present technology, the term ambient will be taken to mean (i) external of the treatment system or patient, and (ii) immediately surrounding the treatment system or patient.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RPT device or emanating from a mask or patient interface. Ambient noise may be generated by sources outside the room.

Automatic Positive Airway Pressure (APAP) therapy: CPAP therapy in which the treatment pressure is automatically adjustable, e.g. from breath to breath, between minimum and maximum limits, depending on the presence or absence of indications of SDB events.

Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction, and decreased in the absence of indications of partial upper airway obstruction.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

In the example of patient respiration, a flow rate may be nominally positive for the inspiratory portion of a breathing cycle of a patient, and hence negative for the expiratory portion of the breathing cycle of a patient. Device flow rate, Qd, is the flow rate of air leaving the RPT device. Total flow rate, Qt, is the flow rate of air and any supplementary gas reaching the patient interface via the air circuit. Vent flow rate, Qv, is the flow rate of air leaving a vent to allow washout of exhaled gases. Leak flow rate, Ql, is the flow rate of leak from a patient interface system or elsewhere. Respiratory flow rate, Qr, is the flow rate of air that is received into the patient's respiratory system.

Flow therapy: Respiratory therapy comprising the delivery of a flow of air to an entrance to the airways at a controlled flow rate referred to as the treatment flow rate that is typically positive throughout the patient's breathing cycle.

Humidifier: The word humidifier will be taken to mean a humidifying apparatus constructed and arranged, or configured with a physical structure to be capable of providing a therapeutically beneficial amount of water (H₂O) vapour to a flow of air to ameliorate a medical respiratory condition of a patient.

Leak: The word leak will be taken to be an unintended flow of air. In one example, leak may occur as the result of an incomplete seal between a mask and a patient's face. In another example leak may occur in a swivel elbow to the ambient.

Oxygen enriched air: Air with a concentration of oxygen greater than that of atmospheric air (21%), for example at least about 50% oxygen, at least about 60% oxygen, at least about 70% oxygen, at least about 80% oxygen, at least about 90% oxygen, at least about 95% oxygen, at least about 98% oxygen, or at least about 99% oxygen. “Oxygen enriched air” is sometimes shortened to “oxygen”.

Medical Oxygen: Medical oxygen is defined as oxygen enriched air with an oxygen concentration of 80% or greater. Patient: A person, whether or not they are suffering from a respiratory condition.

Pressure: Force per unit area. Pressure may be expressed in a range of units, including cmH₂O, g-f/cm² and hectopascal. 1 cmH₂O is equal to 1 g-f/cm² and is approximately 0.98 hectopascal (1 hectopascal=100 Pa=100 N/m²=1 millibar ~0.001 atm). In this specification, unless otherwise stated, pressure is given in units of cmH₂O.

The pressure in the patient interface is given the symbol Pm, while the treatment pressure, which represents a target value to be achieved by the interface pressure Pm at the current instant of time, is given the symbol Pt.

Respiratory Pressure Therapy: The application of a supply of air to an entrance to the airways at a treatment pressure that is typically positive with respect to atmosphere.

Ventilator: A mechanical device that provides pressure support to a patient to perform some or all of the work of breathing.

5.6.1.1 Materials & their Properties

Hardness: Refers to durometer or indentation hardness, which is a material property measured by indentation of an indentor (e.g., as measured in accordance with ASTM D2240).
‘Soft’ materials may include silicone or thermo-plastic elastomer (TPE), and may, e.g. readily deform under finger pressure.
‘Hard’ materials may include polycarbonate, polypropylene, and may not e.g. readily deform under finger pressure.

Silicone or Silicone Elastomer: A synthetic rubber. In this specification, a reference to silicone is a reference to liquid silicone rubber (LSR) or a compression moulded silicone rubber (CMSR). One form of commercially available LSR is SILASTIC (included in the range of products sold under this trademark), manufactured by Dow Corning. Another manufacturer of LSR is Wacker. Unless otherwise specified to the contrary, an exemplary form of LSR has a Shore A (or Type A) indentation hardness in the range of about 35 to about 45 as measured using ASTM D2240.

Polycarbonate: a thermoplastic polymer of Bisphenol-A Carbonate.

### 5.6.1.2 Mechanics

### Axes:

i) Neutral axis: An axis in the cross-section of a beam or plate along which there are no longitudinal stresses or strains.
ii) Longitudinal axis: An axis extending along the length of a shape. The axis generally passes through a center of the shape.
iii) Circumferential axis: An axis oriented perpendicularly with respect to the longitudinal axis. The axis may be specifically present in pipes, tubes, cylinders, or similar shapes with a circular and/or elliptical cross section.

Deformation: The process where the original geometry of a member changes when subjected to forces, e.g. a force in a direction with respect to an axis. The process may include stretching or compressing, bending and, twisting.

Elasticity: The ability of a material to return to its original geometry after deformation.

Floppy structure or component: A structure or component that will change shape, e.g. bend, when caused to support its own weight, within a relatively short period of time such as 1 second.

Resilience: Ability of a material to absorb energy when deformed elastically and to release the energy upon unloading.

Resilient: Will release substantially all of the energy when unloaded. Includes e.g. certain silicones, and thermoplastic elastomers.

Rigid structure or component: A structure or component that will not substantially change shape when subject to the loads typically encountered in use. An example of such a use may be setting up and maintaining a patient interface in sealing relationship with an entrance to a patient's airways, e.g. at a load of approximately 20 to 30 cmH2O pressure.

As an example, an I-beam may comprise a different bending stiffness (resistance to a bending load) in a first direction in comparison to a second, orthogonal direction. In another example, a structure or component may be floppy in a first direction and rigid in a second direction.

Stiffness (or rigidity) of a structure or component: The ability of the structure or component to resist deformation in response to an applied load. The load may be a force or a moment, e.g. compression, tension, bending or torsion. The structure or component may offer different resistances in different directions. The inverse of stiffness is flexibility.

Visco-elasticity: The ability of a material to display both elastic and viscous behaviour in deformation.

Yield: The situation when a material can no longer return back to its original geometry after deformation.

### 5.6.1.3 Structural Elements

Compression member: A structural element that resists compression forces.

Elbow: An elbow is an example of a structure that directs an axis of flow of air travelling therethrough to change direction through an angle. In one form, the angle may be approximately 90 degrees. In another form, the angle may be more, or less than 90 degrees. The elbow may have an approximately circular cross-section. In another form the elbow may have an oval or a rectangular cross-section. In certain forms an elbow may be rotatable with respect to a mating component, e.g. about 360 degrees. In certain forms an elbow may be removable from a mating component, e.g. via a snap connection. In certain forms, an elbow may be assembled to a mating component via a one-time snap during manufacture, but not removable by a patient.

Frame: Frame will be taken to mean a mask structure that bears the load of tension between two or more points of connection with a headgear. A mask frame may be a non-airtight load bearing structure in the mask. However, some forms of mask frame may also be air-tight.

Membrane: Membrane will be taken to mean a typically thin element that has, preferably, substantially no resistance to bending, but has resistance to being stretched.

Tie (noun): A structure designed to resist tension.

### Thin Structures:

a. Beams,
   i. A beam may be relatively long in one dimension compared to the other two dimensions such that the smaller dimensions are comparatively thin compared to the long dimension
b. Membranes,
   i. Relatively long in two dimensions, with one thin dimension. Readily deforms in response to bending forces. Resists being stretched, (might also resist compression).
c. Plates & Shells
   i. These may be relatively long in two directions, with one thin dimension. They may have bending, tensile, and/or compressive stiffness.

### Thick Structures: Solids

Seal: May be a noun form (“a seal”) which refers to a structure, or a verb form (“to seal”) which refers to the effect. Two elements may be constructed and/or arranged to ‘seal’ or to effect ‘sealing’ therebetween without requiring a separate ‘seal’ element per se.

Shell: A shell will be taken to mean a curved, relatively thin structure having bending, tensile and compressive stiffness. For example, a curved structural wall of a mask may be a shell. In some forms, a shell may be faceted. In some forms a shell may be airtight. In some forms a shell may not be airtight.

Stiffener: A stiffener will be taken to mean a structural component designed to increase the bending resistance of another component in at least one direction.

Strut: A strut will be taken to be a structural component designed to increase the compression resistance of another component in at least one direction.

Swivel (noun): A subassembly of components configured to rotate about a common axis, preferably independently, preferably under low torque. In one form, the swivel may be constructed to rotate through an angle of at least 360 degrees. In another form, the swivel may be constructed to rotate through an angle less than 360 degrees. When used in the context of an air delivery conduit, the sub-assembly of components preferably comprises a matched pair of cylindrical conduits. There may be little or no leak flow of air from the swivel in use.

### 5.6.2 Anatomy

### 5.6.2.1 Anatomy of the Face

Ala: the external outer wall or “wing” of each nostril (plural: alar)

Alar angle: An angle formed between the ala of each nostril.

Alare: The most lateral point on the nasal ala.

Alar curvature (or alar crest) point: The most posterior point in the curved base line of each ala, found in the crease formed by the union of the ala with the cheek.

Auricle: The whole external visible part of the ear.

(nose) Bony framework: The bony framework of the nose comprises the nasal bones, the frontal process of the maxillae and the nasal part of the frontal bone.

(nose) Cartilaginous framework: The cartilaginous framework of the nose comprises the septal, lateral, major and minor cartilages.

Columella: the strip of skin that separates the nares and which runs from the pronasale to the upper lip.

Columella angle: The angle between the line drawn through the midpoint of the nostril aperture and a line drawn perpendicular to the Frankfort horizontal while intersecting subnasale.

Frankfort horizontal plane: A line extending from the most inferior point of the orbital margin to the left tragion. The tragion is the deepest point in the notch superior to the tragus of the auricle.

Glabella: Located on the soft tissue, the most prominent point in the midsagittal plane of the forehead.

Lateral nasal cartilage: A generally triangular plate of cartilage. Its superior margin is attached to the nasal bone and frontal process of the maxilla, and its inferior margin is connected to the greater alar cartilage.

Lip, lower (labrale inferius): The lip extending between the subnasale and the mouth.

Lip, upper (labrale superius): The lip extending between the mouth and the supramenton.

Greater alar cartilage: A plate of cartilage lying below the lateral nasal cartilage. It is curved around the anterior part of the naris. Its posterior end is connected to the frontal process of the maxilla by a tough fibrous membrane containing three or four minor cartilages of the ala.

Nares (Nostrils): Approximately ellipsoidal apertures forming the entrance to the nasal cavity. The singular form of nares is naris (nostril). The nares are separated by the nasal septum.

Naso-labial sulcus or Naso-labial fold: The skin fold or groove that runs from each side of the nose to the corners of the mouth, separating the cheeks from the upper lip.

Naso-labial angle: The angle between the columella and the upper lip, while intersecting subnasale.

Otobasion inferior: The lowest point of attachment of the auricle to the skin of the face.

Otobasion superior: The highest point of attachment of the auricle to the skin of the face.

Pronasale: the most protruded point or tip of the nose, which can be identified in lateral view of the rest of the portion of the head.

Philtrum: the midline groove that runs from lower border of the nasal septum to the top of the lip in the upper lip region.

Pogonion: Located on the soft tissue, the most anterior midpoint of the chin.

Ridge (nasal): The nasal ridge is the midline prominence of the nose, extending from the Sellion to the Pronasale.

Sagittal plane: A vertical plane that passes from anterior (front) to posterior (rear). The midsagittal plane is a sagittal plane that divides the body into right and left halves.

Sellion: Located on the soft tissue, the most concave point overlying the area of the frontonasal suture.

Septal cartilage (nasal): The nasal septal cartilage forms part of the septum and divides the front part of the nasal cavity.

Subalare: The point at the lower margin of the alar base, where the alar base joins with the skin of the superior (upper) lip.

Subnasal point: Located on the soft tissue, the point at which the columella merges with the upper lip in the midsagittal plane.

Supramenton: The point of greatest concavity in the midline of the lower lip between labrale inferius and soft tissue pogonion

### 5.6.2.2 Anatomy of the Skull

Frontal bone: The frontal bone includes a large vertical portion, the squama frontalis, corresponding to the region known as the forehead.

Mandible: The mandible forms the lower jaw. The mental protuberance is the bony protuberance of the jaw that forms the chin.

Maxilla: The maxilla forms the upper jaw and is located above the mandible and below the orbits. The frontal process of the maxilla projects upwards by the side of the nose, and forms part of its lateral boundary.

Nasal bones: The nasal bones are two small oblong bones, varying in size and form in different individuals; they are placed side by side at the middle and upper part of the face, and form, by their junction, the “bridge” of the nose.

Nasion: The intersection of the frontal bone and the two nasal bones, a depressed area directly between the eyes and superior to the bridge of the nose.

Occipital bone: The occipital bone is situated at the back and lower part of the cranium. It includes an oval aperture, the foramen magnum, through which the cranial cavity communicates with the vertebral canal. The curved plate behind the foramen magnum is the squama occipitalis.

Orbit: The bony cavity in the skull to contain the eyeball.

Parietal bones: The parietal bones are the bones that, when joined together, form the roof and sides of the cranium.

Temporal bones: The temporal bones are situated on the bases and sides of the skull, and support that part of the face known as the temple.

Zygomatic bones: The face includes two zygomatic bones, located in the upper and lateral parts of the face and forming the prominence of the cheek.

### 5.6.2.3 Anatomy of the Respiratory System

Diaphragm: A sheet of muscle that extends across the bottom of the rib cage. The diaphragm separates the thoracic cavity, containing the heart, lungs and ribs, from the abdominal cavity. As the diaphragm contracts the volume of the thoracic cavity increases and air is drawn into the lungs.

Larynx: The larynx, or voice box houses the vocal folds and connects the inferior part of the pharynx (hypopharynx) with the trachea.

Lungs: The organs of respiration in humans. The conducting zone of the lungs contains the trachea, the bronchi, the bronchioles, and the terminal bronchioles. The respiratory zone contains the respiratory bronchioles, the alveolar ducts, and the alveoli.

Nasal cavity: The nasal cavity (or nasal fossa) is a large air filled space above and behind the nose in the middle of the face. The nasal cavity is divided in two by a vertical fin called the nasal septum. On the sides of the nasal cavity are three horizontal outgrowths called nasal conchae (singular “concha”) or turbinates. To the front of the nasal cavity is the nose, while the back blends, via the choanae, into the nasopharynx.

Pharynx: The part of the throat situated immediately inferior to (below) the nasal cavity, and superior to the oesophagus and larynx. The pharynx is conventionally divided into three sections: the nasopharynx (epipharynx) (the nasal part of the pharynx), the oropharynx (mesopharynx) (the oral part of the pharynx), and the laryngopharynx (hypopharynx).

### 5.6.3 Patient Interface

Anti-asphyxia valve (AAV): The component or sub-assembly of a mask system that, by opening to atmosphere in a failsafe manner, reduces the risk of excessive CO2 rebreathing by a patient.

Headgear: Headgear will be taken to mean a form of positioning and stabilising structure designed to hold a device, e.g., a mask, on a head.

Plenum chamber: a mask plenum chamber will be taken to mean a portion of a patient interface having walls at least partially enclosing a volume of space, the volume having air therein pressurised above atmospheric pressure in use. A shell may form part of the walls of a mask plenum chamber.

Seal: May be a noun form (“a seal”) which refers to a structure, or a verb form (“to seal”) which refers to the effect. Two elements may be constructed and/or arranged to ‘seal’ or to effect ‘sealing’ therebetween without requiring a separate ‘seal’ element per se.

Vent (noun): A structure that allows a flow of air from an interior of the mask, or conduit, to ambient air for clinically effective washout of exhaled gases. For example, a clinically effective washout may involve a flow rate of about 10 litres per minute to about 100 litres per minute, depending on the mask design and treatment pressure.

### 5.6.4 Shape of Structures

Products in accordance with the present technology may comprise one or more three-dimensional mechanical structures, for example a mask cushion or an impeller. The three-dimensional structures may be bounded by two-dimensional surfaces. These surfaces may be distinguished using a label to describe an associated surface orientation, location, function, or some other characteristic. For example a structure may comprise one or more of an anterior surface, a posterior surface, an interior surface and an exterior surface. In another example, a seal-forming structure may comprise a face-contacting (e.g. outer) surface, and a separate non-face-contacting (e.g. underside or inner) surface. In another example, a structure may comprise a first surface and a second surface.

### 5.7 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Furthermore, “approximately”, “substantially”, “about”, or any similar term used herein means+/−5-10% of the recited value.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

5.8 REFERENCE SIGNS LIST

| | |
|---|---|
| patient | 1000 |
| bed partner | 1100 |
| patient interface | 3000 |
| seal - forming structure | 3100 |
| cushion module | 3150 |
| plenum chamber | 3200 |
| positioning and stabilising structure | 3300 |
| strap | 3310 |
| tab | 3320 |
| conduit tube | 3350 |
| first textile layer (of conduit tube) | 3352 |
| second textile layer (of conduit tube) | 3354 |
| hollow interior | 3356 |
| extendable tube sections | 3362 |
| non - extendable tube sections | 3363 |
| outer surface | 3380 |
| inner surface | 3382 |
| vent | 3400 |
| connection port | 3600 |
| elbow | 3610 |
| RPT device | 4000 |
| humidifier | 5000 |

1.-31. (canceled)

32. A conduit tube for use in an air circuit that connects a respiratory pressure therapy (RPT) device which is configured to generate a flow of air at a positive pressure above ambient, and a patient interface configured to receive the flow of air, and deliver the flow of air to one or more airways of a patient for treatment of a respiratory disorder, wherein the conduit tube has a length, an external circumference, and an internal circumference, and wherein the conduit has a hollow interior for the flow of air to pass therethrough from the RPT device to the patient interface, wherein the hollow interior is defined by a layer of impermeable material, the conduit tube comprising:
an outer textile layer, wherein the outer textile layer is continuous along the length and external circumference of the conduit tube; and
an inner layer that forms the layer of impermeable material, wherein the inner layer permeates at least partially into the outer textile layer, and wherein the layer of impermeable material is continuous along the length and internal circumference of the conduit tube.

33. A conduit tube for use in an air circuit that connects a respiratory pressure therapy (RPT) device which is configured to generate a flow of air at a positive pressure above ambient, and a patient interface configured to receive the flow of air, and deliver the flow of air to one or more airways of a patient for treatment of a respiratory disorder, wherein the conduit tube has a length, an external circumference, and an internal circumference, and wherein the conduit has a hollow interior for the flow of air to pass therethrough from the RPT device to the patient interface, wherein the hollow interior is defined by a layer of impermeable material, the conduit tube comprising:
an outer textile layer, wherein the outer textile layer is continuous along the length and external circumference of the conduit tube; and
an inner layer that forms the layer of impermeable material, wherein the inner layer comprises a phase change material, and wherein the inner layer is applied to the outer textile layer in a first phase condition and is bonded to the outer textile layer in a second phase condition, and wherein in the second phase condition, the layer of impermeable material is continuous along the length and internal circumference of the conduit tube.

34. The conduit tube as claimed in claim 32, wherein the outer textile layer comprises a plurality of loops of yarn in a knitted structure.

35. The conduit tube as claimed in claim 32, wherein the inner layer has a contact surface and an inner surface, the inner surface defining the hollow interior of the conduit tube.

36. The conduit tube as claimed in claim 32, wherein the inner layer is a polymer.

37. The conduit tube as claimed in claim 32, wherein the conduit tube is seamless.

38. A conduit tube for use in an air circuit that connects a respiratory pressure therapy (RPT) device which is configured to generate a flow of air at a positive pressure above ambient, and a patient interface configured to receive the flow of air, and deliver the flow of air to one or more airways of a patient for treatment of a respiratory disorder, wherein the conduit tube has a length, an external circumference, and an internal circumference, and wherein the conduit has a hollow interior for the flow of air to pass therethrough from the RPT device to the patient interface, wherein the hollow interior is defined by a layer of impermeable material, the conduit tube comprising:
a first textile layer, wherein the first textile layer is continuous along the length and external circumference of the conduit tube; and
a second textile layer, wherein the second textile layer is knitted to the first textile layer and forms the layer of impermeable material of the hollow interior of the conduit tube, and wherein the layer of impermeable material is continuous along the length and internal circumference of the conduit tube.

39. The conduit tube as claimed in claim 38, wherein the first textile layer forms at least part of the external circumference or a patient contacting side of the conduit tube.

40. The conduit tube as claimed in claim 38, wherein the first textile layer is a breathable textile.

41. The conduit tube as claimed in claim 38, wherein the first textile layer is knitted to the second textile layer to form a plated knit structure comprising a technical face and a technical back, wherein the technical face is formed by a first yarn comprising the first textile layer, and the technical back is formed by a second yarn comprising the second textile layer, and wherein the first textile layer and the second textile layer is knitted together.

42. The conduit tube as claimed in claim 41, wherein the plated knit structure comprises one or more yarns as a series of loops.

43. The conduit tube as claimed in claim 41, wherein the yarn of the first textile layer is one of a synthetic yarn, a natural yarn, or a blend thereof.

44. The conduit tube as claimed in claim 43, wherein the yarn of the first textile layer comprises a polyamide material.

45. The conduit tube as claimed in claim 44, wherein the first textile layer comprises a yarn of nylon.

46. The conduit tube as claimed in claim 43, wherein the yarn of the first textile layer comprises a polyurethane material.

47. The conduit tube as claimed in claim 46, wherein the first textile layer comprises a yarn of a polyurethane co-polymer material.

48. The conduit tube as claimed in claim 47, wherein the first textile layer comprises a yarn of elastane.

49. The conduit tube as claimed in claim 38, wherein the yarn of the second textile layer is a phase changing polymer yarn.

50. The conduit tube as claimed in claim 49, wherein the yarn of the second textile layer is one of polypropylene, polyethylene, thermoplastic polyurethane, and a thermoplastic elastomer, or a blend thereof.

51. The conduit tube as claimed in claim 38, wherein the second textile layer undergoes a phase change during manufacture of the conduit tube.

52. The conduit tube as claimed in claim 51, wherein the phase change is stimulated or otherwise initiated through application of heat, chemicals, or a mechanical process.

53. The conduit tube as claimed in claim 38, wherein the conduit tube is seamless.

54. A method of manufacturing a conduit tube for use in an air circuit that connects a respiratory pressure therapy (RPT) device which is configured to generate a flow of air at a positive pressure above ambient, and a patient interface configured to receive the flow of air, and deliver the flow of air to one or more airways of a patient for treatment of a respiratory disorder, wherein the conduit tube has a length, an external circumference, and an internal circumference, and wherein the conduit has a hollow interior for the flow of air to pass therethrough from the RPT device to the patient interface, wherein the hollow interior is defined by a layer of impermeable material, the method comprising the following steps:
a) forming an outer textile layer, wherein the outer textile layer is continuous along the length and external circumference of the conduit tube and has an inner surface;
b) applying the layer of impermeable material to the inner surface of the outer textile layer, wherein the layer of impermeable material is continuous along the length and internal circumference of the conduit tube; and
c) implementing a phase change in the layer of impermeable material such that it permeates at least partially into the outer textile layer.

55. The method as claimed in claim 54, wherein the method includes a further step of implementing a second phase change in the layer of impermeable material to bond it to the outer textile layer.

56. A method of manufacturing a conduit tube for use in an air circuit that connects a respiratory pressure therapy (RPT) device which is configured to generate a flow of air at a positive pressure above ambient, and a patient interface configured to receive the flow of air, and deliver the flow of air to one or more airways of a patient for treatment of a respiratory disorder, wherein the conduit tube has a length, an external circumference, and an internal circumference, and wherein the conduit has a hollow interior for the flow of air to pass therethrough from the RPT device to the patient interface, the method comprising the following steps:
a) providing a textile layer which has a first surface that, in use, forms a continuous outer surface along the length and external circumference of the conduit tube;
b) applying a phase change material to a second surface of the textile layer, wherein the phase change material is in a first phase condition; and
c) implementing a change in the phase change material from the first phase condition to a second phase condition, wherein, when the phase change material is in the second phase condition, the phase change material forms a layer of impermeable material defining the hollow interior of the conduit tube, and wherein in the second phase condition, the layer of impermeable material is continuous along the length and internal circumference of the conduit tube.

57. A method of manufacturing a conduit tube for use in an air circuit that connects a respiratory pressure therapy (RPT) device which is configured to generate a flow of air at a positive pressure above ambient, and a patient interface configured to receive the flow of air, and deliver the flow of air to one or more airways of a patient for treatment of a respiratory disorder, wherein the conduit tube has a continuous length, external circumference, and internal circumference, and wherein the conduit tube has a hollow interior for the flow of air to pass therethrough from the RPT device to the patient interface, the method comprising the following steps:
a) knitting a first yarn to a second yarn to form a plated knit structure, having a technical face and a technical back;
b) implementing a first phase change of the first yarn; and
c) implementing a second phase change of the first yarn.

58. The method as claimed in claim 57, wherein step b) comprises applying a chemical agent to at least a portion of the first yarn dominating the technical back to stimulate the first phase change from a solid to at least a semi-solid.

59. The method as claimed in claim 58, wherein step c) comprises applying a neutralising agent to at least a portion of the first yarn dominating the technical back to stimulate the second phase change from at least a semi-solid to a solid.

60. The method as claimed in claim 58, wherein step c) comprises effecting a change in a temperature of the portion of the first yarn dominating the technical back to stimulate the second phase change from at least a semi-solid to a solid.

61. The method as claimed in claim 57, wherein step b) comprises positioning the plated knit structure on a shaping component with a predefined shape, e.g. a mould or a mandrel with a predefined shape.

62. The method as claimed in claim 61, wherein step b) also comprises the plated knit structure being heated by the shaping component to a temperature at which the first yarn may undergo at least a first partial phase change for a time period.

63. The method as claimed in claim 61, wherein step c) comprises cooling the shaping component to a temperature at which the first yarn may undergo at least a second partial phase change, allowing the first yarn to at least partially integrate and bond to the second yarn.

64. The method as claimed in claim 62, wherein step c) comprises the knitted plate structure being cooled by the shaping component to a temperature at which the second yarn will set/harden to a solid, and in doing so bond with the first yarn.
